(19) United States
(12) Patent Application Publication
(10) Pub. No.: US 2017/0003291 A1
(43) Pub. Date: Jan. 5, 2017

(54) METHODS FOR DETECTING, DIAGNOSING AND TREATING ENDOMETRIAL CANCER

(71) Applicant: William Beaumont Hospital, Royal Oak, MI (US)

(72) Inventor: Ray Bahado-Singh, Grosse Pointe Shores, MI (US)

(21) Appl. No.: 15/191,631

(22) Filed: Jun. 24, 2016

Related U.S. Application Data
(60) Provisional application No. 62/185,602, filed on Jun. 27, 2015.

(51) Int. Cl. G01N 33/574; A61B 8/08; A61B 5/00

(52) U.S. Cl. CPC G01N 33/57442; G01N 33/57488; A61B 5/4325; A61B 8/0858; G01N 2570/00; G01N 2405/00; G01N 2405/04

(57) ABSTRACT

The present invention relates to methods for detecting, diagnosing and/or treating endometrial cancer by detecting in a biological sample from a patient the levels of one or more of the metabolites: C14.2, PC ae C38:1, 3-Hydroxybutyric acid, C18:2, PC ae C40:1, and C6 (C4:1-DC). In some embodiments, the method also includes diagnosing the patient with endometrial cancer when the one or more metabolites in the biological sample is at a different level than a statistically validated threshold for the one or more metabolites, and ultrasound indicates endometrial cancer in the patient. In further embodiments, once endometrial cancer is diagnosed, the patient is treated for the endometrial cancer.

### CROSS REFERENCE TO RELATED APPLICATION

This application claims priority to U.S. Ser. No. 62/185,602, filed Jun. 27, 2015. The entire contents of the aforementioned application are incorporated herein.

### FIELD OF THE INVENTION

The present invention is in the field of biochemistry and medicine and relates to methods for detecting, diagnosing, and/or treating endometrial cancer.

### BACKGROUND OF THE INVENTION

Metabolomics is the newest member of the “omics” systems biology discipline. In recent times there has been an explosion of publications related to the use of metabolomics for the analysis of complex disorders. Cancer analytics has been a principal focus. Applications and areas of promise for cancer metabolomics include early detection and disease staging. Further, metabolomics also has the potential to identify individuals who are likely to respond to particular cancer therapies (individualized medicine) and has proven of value in determining the effects of therapeutic agents on cancer cells.

So far, few studies in the literature have addressed the metabolomics of gynecologic cancers. In such studies, the focus has mainly been on ovarian. Based on the U.S. national cancer database Surveillance Epidemiology and End Results (SEERS) figures, uterine cancer is the most common gynecologic malignancy in the U.S.A. Moreover, the incidence in the U.S. is higher than for other developed countries. Metabolomic interrogation of endometrial cancer (EC) is an area of potential scientific and clinical interest.

There is significant current interest in biomarkers for endometrial cancer (EC). Biomarkers are needed to predict disease spread, prognosis and for individualizing treatment strategies. Further there is great interest in predicting which patients might relapse. Significant advances remain to be achieved in each of these domains. The predominant interest in the literature up to now has been molecular biomarkers (e.g. mutations of the phosphatase and tensin homolog (PTEN) tumor suppressor gene). There remains substantial interest in serum protein biomarkers such as CA125, HE4, and growth differentiating factor (GDF) for determining disease spread. Metabolomic fluctuations reflect alterations in the genome, epigenome, transcriptome and proteome thus providing information on molecular changes and more. Metabolomics provides significant details of cell function and disorder that exceeds that provided by more established analytic methods such as genomics and proteomics. As a consequence, metabolomics is now regarded as a powerful tool for cancer diagnosis and for the discovery of novel biomarkers.

Metabolomic studies have confirmed that cancer is a metabolic disorder with profound alterations of critical pathways such as glycolysis, tricarboxylic acid cycle, choline and fatty acid metabolism in cancer cells. Metabolomics has now been successfully utilized for biomarker development in most major cancers breast, lung, colorectal and prostate cancers.

Gaudet et al. reported that five metabolites isovalerylcarnitine/2-methylbutylcarnitine, ocenoylcarnitine, linoleic acid, decatrienolycarnitine, and stearic acid correlate with the diagnosis of EC (however, the latter two, decatrienolycarnitine, and stearic acid, were not found to be significant when adjusted for clinical confounders).

### SUMMARY OF THE INVENTION

Metabolomics can be used for the detection of metabolite changes even at micromolar concentrations. The inventors were able to use this tool to identify metabolic biomarkers of endometrial cancer. This reported measurement of metabolites can be applied to any body fluid (blood, urine, saliva, breath condensate, cervico-vagina fluid) and hair or nail samples.

Using combined NMR and MS metabolomic analysis the inventors found statistically significant changes in the serum metabolome of patients diagnosed with EC compared with unaffected controls (normal). Of the total of 181 metabolites evaluated, four metabolites using NMR and fifty-three metabolites using Mass spectrometry showed significant changes in concentration in EC versus normals. Further, a combination of three metabolomic markers predicted the presence of EC with good diagnostic accuracy, AUC >0.80. Due to the significantly improved prognosis when EC is confined to the uterus, the inventors also investigated whether metabolomic markers could significantly distinguish early stage disease endometrial cancer, confined to the uterus (FIGO stages I and II) from unaffected patients. The metabolite algorithm achieved good diagnostic accuracy AUC >0.80.

In one aspect, disclosed is a method of detecting a level of two or more metabolites in a biological sample, where the method consists of obtaining a biological sample from a human patient, where the biological sample includes two or more of C14.2, PC ae C38:1, 3-Hydroxybutyric acid, C18:2, PC ae C40:1, and C6 (C4:1-DC); and detecting the level of the two or more metabolites in the biological sample. In some aspects, the sample may be blood serum. In other aspects of the invention, the two or more metabolites may be C14.2, PC ae C38:1, and 3-Hydroxybutyric acid; or the two or more metabolites may be C18:2, PC ae C40:1, and C6 (C4:1-DC).

In other aspects of the invention, the level of the two or more metabolites may be detected by performing nuclear magnetic resonance (NMR) or mass spectrometry (MS) on the biological sample; or the two or more metabolites may be detected by magnetic resonance spectroscopy (MRS) or proton magnetic resonance spectroscopy (1H-MRS) assessed using magnetic resonance imaging (MRI).

In another aspect, the inventive method is diagnosing endometrial cancer in a human patient, wherein the patient has a uterus with an endometrium, and method includes obtaining a biological sample from the human patient, where the biological sample includes one or more metabolites C14.2, PC ae C38:1, 3-Hydroxybutyric acid, C18:2, PC ae C40:1, and C6 (C4:1-DC); detecting a level of the one or more metabolites in the biological sample; performing ultrasound on the uterus of the patient to measure the thickness of the endometrium of the patient; and diagnosing the patient with endometrial cancer when (a) the one or more metabolites in the biological sample is at a different level than a statistically validated threshold for the one or more metabolites and (b) the ultrasound indicates endometrial cancer in the patient.

In other aspects of the invention, the level of the one or more metabolites may be detected by performing nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) by using MRI. The sample may be blood serum; the one or more metabolites may be C14.2, PC ae C38:1, and 3-Hydroxybutyric acid; or the one or more metabolites may be C18:2, PC ae C40:1, and C6 (C4:1-DC).

A further aspect is a method of diagnosing and treating endometrial cancer in a subject, the method comprising: obtaining a biological sample from the human subject, where the biological sample includes one or more metabolites C14.2, PC ae C38:1, 3-Hydroxybutyric acid, C18:2, PC ae C40:1, and C6 (C4:1-DC); detecting a level of the one or more metabolites in the biological sample; diagnosing the subject with endometrial cancer when the one or more metabolites in the biological sample is at a different level than a statistically validated threshold for the one or more metabolites; and administering a therapeutically effective amount of a treatment for endometrial cancer to the diagnosed subject. In other aspects of this invention, the level of the one or more metabolites may be detected by performing magnetic resonance imaging (MRI), nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) assessed by using MRI. The sample may be blood serum; the one or more metabolites may be C14.2, PC ae C38:1, and 3-Hydroxybutyric acid; or the one or more metabolites may be C18:2, PC ae C40:1, and C6 (C4:1-DC).

In one embodiment, the present inventive method includes providing medical services for a human patient suspected of having or having endometrial cancer, this method including requesting a biological sample from and diagnostic information about the patient, where the diagnostic information is a level of one or more metabolites C14.2, PC ae C38:1, 3-Hydroxybutyric acid, C18:2, PC ae C40:1, and C6 (C4:1-DC) in the biological sample; and administering a therapeutically effective amount of a treatment for endometrial cancer when the diagnostic information indicates that the level of the one or more metabolites in the biological sample is at a different level than a statistically validated threshold for the one or more metabolites. In other aspects of this invention, the level of the one or more metabolites may be detected by performing nuclear magnetic resonance (NMR) or mass spectrometry (MS) on the biological sample; or the one or more metabolites may be detected by magnetic resonance spectroscopy (MRS) or proton magnetic resonance spectroscopy (1H-MRS) by using MRI. The sample may be blood serum; the one or more metabolites may be C14.2, PC ae C38:1, and 3-Hydroxybutyric acid; or the one or more metabolites may be C18:2, PC ae C40:1, and C6 (C4:1-DC).

Another embodiment of the present invention is a method of monitoring treatment for endometrial cancer in a human patient, comprising: requesting a first biological sample from and first diagnostic information about the patient, wherein the first diagnostic information is a level of one or more metabolites C14.2, PC ae C38:1, 3-Hydroxybutyric acid, C18:2, PC ae C40:1, and C6 (C4:1-DC) in the first biological sample; administering a therapeutically effective amount of a treatment for endometrial cancer to the patient; after administering the therapeutically effective amount of the treatment for endometrial cancer to the patient, requesting a second biological sample from and second diagnostic information about the patient, wherein the second diagnostic information is a level of one or more C14.2, PC ae C38:1, 3-Hydroxybutyric acid, C18:2, PC ae C40:1, and C6 (C4:1-DC) in the second biological sample; and comparing the first diagnostic information and the second diagnostic information to determine whether the level of the one or more metabolites in the first biological sample is at a different level than the level of the one or more metabolites in the second biological sample. In a further embodiment, the sample may be serum. Based on metabolite response after treatment, the risk of developing certain complications can be predicted. Further, the patient's metabolite profile may be performed before treatment and, based on the concentrations of certain metabolites, the likelihood of successful response can be estimated prior to actual therapy.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIG. 1A is a 2D Principal Component Analysis (PCA) plot showing Endometrial Cancer versus Normal Controls. FIG. 1B is a 3D PCA plot showing Endometrial Cancer versus Normal Controls. (Data log-transformed, Pareto Scaling used.)

FIG. 2A is a 2D Partial Least Square Discriminant analysis (PLS-DA) plot showing Endometrial Cancer versus Normal Controls. FIG. 2B is a 3D PLS-DA plot showing Endometrial Cancer versus Normal. (Data log-transformed, Pareto Scaling used.)

FIG. 3 is a Variable Importance in Projection Plot (VIP) plot showing: Endometrial Cancer (all cases) versus Normal Controls. Permutation test (2000 repeats) for the PLS-DA Model: p-value <0.001.

FIG. 4A is a 2D Principal Component Analysis (PCA) plot showing Early Endometrial Cancer versus Normal Controls. FIG. 4B is a 3D PCA plot showing Early Endometrial Cancer versus Normal Controls.

FIG. 5 is a Variable Importance in Projection Plot (VIP) plot showing: Early Endometrial Cancer versus Normal Controls. Permutation test (2000 repeats) for the PLS-DA Model: p-value <0.001.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well-known and commonly employed in the art. Although any methods, devices and materials similar or equivalent to those described herein can be used in the practice or testing of the invention, the inventive methods, devices and materials are now described.

### DEFINITIONS

In this specification and the appended claims, the singular forms “a,” “an” and “the” include plural reference unless the context clearly dictates otherwise.

As used in the application, “administering”, when used in conjunction with a treatment means providing or performing medical services with respect to a subject in need of a treatment. For example, when used when used in conjunction with a therapeutic, administering means to deliver a therapeutic directly into or onto a target tissue or to administer a therapeutic to a subject whereby the therapeutic positively impacts the tissue to which it is targeted. “Administering” a composition may be accomplished by oral administration, injection, infusion, absorption or by any method in combination with other known techniques. “Administering” may include the act of self-administration or administration by another person such as, for example, a healthcare provider or other individual.

As used in the present application, “biological sample” means a specimen or culture obtained from any biological source. Biological samples may be obtained from animals (including humans). For example, biological samples may be obtained from a normal subject, a subject suspected of having endometrial cancer, or a subject with endometrial cancer. Biological samples encompass fluids, solids, tissues, gases, and other material derived from a biological organism (e.g., hair or nails). Exemplary fluids include blood products (e.g., whole blood, serum, or plasma) and other fluids typically found within or produced by an organism, such as, cervicovaginal secretions (whether blood stained or otherwise), uterine cavity lavage, sweat, breath condensate, urine, saliva, tears, cerebrospinal fluid, milk, vitreous fluid, amniotic fluid, bile, ascites fluid, pus, and the like. Also included within the meaning of the term “biological sample” is an organ or tissue extract (e.g., endometrial tissue, tumor tissue, biopsy specimens) and culture fluid in which any cells or tissue preparation from a subject has been incubated. Other material derived from a biological organism includes smoke from the cauterization of EC tumor or normal tissue (and that material can be analyzed for relevant metabolites using MS or NMR).

The terms “diagnosis” or “diagnosing” mean a determination (by one or more individuals) that the cause or nature of a problem, situation, or condition in a subject is endometrial cancer, or a confirmation of the diagnosis of the disease that includes alternative endometrial cancer diagnostics, other signs and/or symptoms (e.g., based in whole or in part on the level(s) of the one or more endometrial cancer-indicating metabolites described herein). A “diagnosis” of endometrial cancer may include a test or an assessment of the degree of disease severity (e.g., “mild,” “moderate,” or “severe”), current state of disease progression (e.g., “early”, “middle,” or “late” stages of endometrial cancer), or include a comparative assessment to an earlier diagnosis (e.g., the endometrial cancer's symptoms are advancing, stable, or in remission). A diagnosis may include a “prognosis,” that is, a future prediction of the progression of endometrial cancer, based on the observed disease state (e.g., based in whole or in part on the different level(s) of the one or more endometrial cancer-indicating metabolites described herein). A diagnosis or prognosis may be based on one or more biological samples obtained from a subject, and may involve a prediction of disease response to a particular treatment or combination of treatments for endometrial cancer.

The term “endometrial cancer” or “EC” means a type of cancer that begins in the uterus. The uterus is the hollow, pear-shaped pelvic organ in women where fetal development occurs. Endometrial cancer begins in the layer of cells that form the lining (endometrium) of the uterus.

The term “subject” or “patient” as used herein generally refers to any living organism to and may include, but is not limited to, any human, primate, or non-human mammal in need of diagnosis and/or treatment for a condition, disorder or disease (e.g., endometrial cancer). A “subject” may or may not be exhibiting the signs, symptoms, or pathology of endometrial cancer at any stage of any embodiment.

The term “therapeutically effective amount” refers to the amount of treatment (e.g., of an active agent or pharmaceutical compound or composition) that elicits a biological and/or medicinal response in a patient, subject, tissue, or system that is being sought by a researcher, veterinarian, medical doctor or other clinician, or any combination thereof. A biological or medicinal response may include, for example, one or more of the following: (1) preventing a disorder, disease, or condition in an individual that may be predisposed to the disorder, disease, or condition but does not yet experience or display pathology or symptoms of the disorder, disease, or condition, (2) inhibiting a disorder, disease, or condition in an individual that is experiencing or displaying the pathology or symptoms of the disorder, disease, or condition or arresting further development of the pathology and/or symptoms of the disorder, disease, or condition, and/or (3) ameliorating a disorder, disease, or condition in an individual that is experiencing or exhibiting the pathology or symptoms of the disorder, disease, or condition or reversing the pathology and/or symptoms disorder, disease, or condition experienced or exhibited by the individual.

The term “treatment” or “treating” as used herein refers to administrating a medicine or the performance of medical procedures with respect to a subject, for either prophylaxis (prevention) or to cure or reduce the extent of or likelihood of occurrence or recurrence of an infirmity or malady or condition or event in the instance where the subject is afflicted. As related to the present invention, the term may also mean administrating medicine or the performance of medical procedures as therapy, prevention or prophylaxis of endometrial cancer.

The inventors' metabolomics analysis using NMR and DI-MS detection platforms, 4 metabolite biomarkers using NMR and 53 metabolites using Mass spectrometry, had significantly different concentrations in EC versus normal and could thus be used as metabolite biomarkers for diagnosis, staging i.e. determining degree of spread including to lymph nodes or prognosticating outcome, response to therapy and recurrence of EC (Tables 4 and 5 below). Further, the inventors derived various efficacious combinations using the following six of those metabolites: C14.2, PC ae C38:1, 3-Hydroxybutyric acid, C18:2, PC ae C40:1, and C6 (C4:1-DC). (See, Table 6 below).

Tetradecadienyl-L-carnitine (C14:2) is an acyl carnitine which participates in the metabolism so called β-oxidation of fatty acids (lipid components). Acyl-carnitines bind fatty acids and transport them across the mitochondrial membranes where the carbon chain is broken down (metabolized) two carbons at a time are chopped off from the carbon chain that constitutes the back-bone of the fatty acid, thus shortening the carbon chain and metabolizing the fatty acid with the generation of energy for cell use. Lipid abnormalities are thought to play a role in EC development.

Phosphatidylcholine acyl-alkyl C 38:1 (PC ae C38:1) is a glycerophosphocholine, that is, an alkyl,acyl-sn-glycero-3-phosphocholine in which the alkyl or acyl groups at positions 1 and 2 contain a total of 38 carbons and 1 double bond. Phosphotidyl cholines are phospholipids that have incorporated cholines as a part of their structure. They are an important component of cell membranes and are available from various food substances such as egg yolk. Phosphatidylcholines are found in the cell membranes of all animal cells.

3-Hydroxybutyric acid (or beta-hydroxybutyrate) is a ketone body. Like the other ketone bodies (acetoacetate and acetone), levels of 3-hydroxybutyrate in blood and urine are raised in ketosis. In humans, 3-hydroxybutyrate is synthesized in the liver from acetyl-CoA, and can be used as an energy source by the brain when blood glucose is low. Ketone bodies including (3-hydroxybutyric acid) serve as an indispensable source of energy for extrahepatic tissues, especially the brain and lung of developing mammals. Another important function of ketone bodies is to provide acetoacetyl-CoA and acetyl-CoA for synthesis of cholesterol, fatty acids, and complex lipids. Lipid abnormalities (e.g., associated with obesity, diabetes and unopposed estrogen use) are known to be associated with an increased risk of endometrial cancer.

C18:2 (Octadecadienyl-L-carnitine) is another acyl carnitine. Acylcarnitine represent the combination of a fatty acid substance with carnitine. Carnitine acts as a shuttle to get the fatty acid across the mitochondrial membranes into the mitochondria proper where it can get metabolized (oxidative metabolism). The fatty acids are metabolized by breaking off two carbons at a time from the long carbon chain of the fatty acid.

Phosphatidylcholine acyl-alkyl C 40:1 (PC ae C40:1): Phosphotidylcholine are phospholipids that contain choline. They are present in significant concentrations of and are important components of cell membranes.

C6 (C4:1-DC) is a hexanoylcarnitine (fumarylcarnitine). This is an acylcarnitine with C16:2 fatty acid moiety. Acylcarnitine is useful in the diagnosis of fatty acid oxidation disorders (disorders in metabolism of fats). Since fatty acid metabolism occurs in the mitochondria, abnormalities in the levels of this metabolite points to the lipid abnormality in EC. Said lipid abnormality is manifested by the fact that obesity is a major risk factor for EC, accounting for an estimated 40-50% of EC in the US and Europe.

One aspect of the invention is a method of detecting a level of one or more, two or more, three or more, four or more, five or more, or as many as 54 metabolites in a biological sample. Four discriminating metabolites were identified using NMR; and 53 discriminating metabolites were identified using Mass spectrometry, however, there were 3 of the same discriminating metabolites identified by both NMR and Mass spectrometry (2-Hyroxybutyrate, L-Methionine and Acetone). This method includes obtaining a biological sample from a human patient, wherein said biological sample has one or more, two or more, three or more, four or more, five or more, of C14.2, PC ae C38:1, 3-Hydroxybutyric acid, C18:2, PC ae C40:1, and C6 (C4:1-DC), or as many as 54 metabolites; and detecting the level of the one or more, two or more, three or more, four or more, five or more, or as many as 54 metabolites in the biological sample. Detection of the metabolites in sample can be performed or ordered as part of the inventive diagnostic methods or the diagnostic and treatment methods described herein.

For example, the methods and assays of the present invention detect one or more metabolites in a biological sample from a subject suspected of having or having endometrial cancer. Some metabolites suitable for detection in this invention include: C14.2, PC ae C38:1, 3-Hydroxybutyric acid, C18:2, PC ae C40:1, and C6 (C4:1-DC), which may be used alone or with other metabolite biomarkers or endometrial cancer diagnostics. Also, metabolites suitable for detection in this invention include any of the 54 metabolites having statistically significant concentration changes as described in the Examples below.

In one embodiment of the invention, each metabolite is considered, evaluated and used individually and separately. In another embodiment of the invention, two metabolites are considered, evaluated and used in combinations of two or more to diagnose endometrial cancer. For example, in one aspect of the invention, the metabolites that are detected are C14.2, PC ae C38:1, 3-Hydroxybutyric acid, C18:2, PC ae C40:1, and C6 (C4:1-DC) (e.g., in a fluid biological sample, such as, serum). In another aspect of the invention, the metabolites that are detected are C14.2, PC ae C38:1, and 3-Hydroxybutyric acid. In another aspect of the invention, the metabolites that are detected are C18:2, PC ae C40:1, and C6 (C4:1-DC).

Further aspects of the invention include detecting any combination of the 54 discriminating metabolites (as described above and in Examples below) having statistically significant concentration changes, combined in any number and any combination, and diagnosing endometrial cancer. In another aspect, the invention includes diagnosing endometrial cancer by detecting any combination of the following six metabolites in a sample: C14.2, PC ae C38:1, 3-Hydroxybutyric acid, C18:2, PC ae C40:1, and C6 (C4:1-DC), combined in any number and any combination. For example, using six metabolites, all metabolite combinations of 2, 3, 4, 5, and 6 metabolites, combined in any number and any combination, can be used. Thus, C14.2 can be used in any combination with any of the following in combinations of 2-5 other metabolites including: PC ae C38:1, 3-Hydroxybutyric acid, C18:2, PC ae C40:1, and C6 (C4:1-DC). Similarly, PC ae C38:1 can be used in any combination with any of the following in combinations of 2-5 including: C14.2, 3-Hydroxybutyric acid, C18:2, PC ae C40:1, and C6 (C4:1-DC). Similarly, 3-Hydroxybutyric acid can be used in any combination with any of the following in combinations of 2-5 including C14.2, PC ae C38:1, C18:2, PC ae C40:1, and C6 (C4:1-DC). Similarly, each of C18:2, PC ae C40:1, and C6 (C4:1-DC) each can be used in combination with 2-5 of the other metabolites.

One aspect of the inventive is a method for diagnosing endometrial cancer in a human patient, where the patient has a uterus with an endometrium and the method includes: obtaining a biological sample from the human patient, wherein said biological sample includes one or more metabolites C14.2, PC ae C38:1, 3-Hydroxybutyric acid, C18:2, PC ae C40:1, and C6 (C4:1-DC); detecting a level of the one or more metabolites in the biological sample; performing an ultrasound on the uterus; and diagnosing the patient with endometrial cancer when (a) the one or more metabolites in the biological sample is at a different level than a statistically validated threshold for the one or more metabolites and (b) the ultrasound indicates endometrial cancer. As otherwise described herein, the different level may be a reduced level or an elevated level.

Methods of obtaining biological samples from a subject suspected of endometrial cancer or having endometrial cancer are well known in the art. The biological sample may include one or more metabolites C14.2, PC ae C38:1, 3-Hydroxybutyric acid, C18:2, PC ae C40:1, and C6 (C4:1-DC). Given the ease and convenience with which appropriate samples for analysis can be collected and analyzed, diagnosis of early stage endometrial cancer, and ongoing surveillance for progression of the cancer, is potentially feasible. In addition, fluids other than blood can be obtained for use in the present inventive methods. That is, the need for a needle-stick to obtain blood for testing could be minimized as other body fluids could also be used for testing. Overall, this approach would reduce healthcare cost, costs due to loss of work time, increase patient convenience and reduce discomfort of sample collection (pelvic exam or blood draw) and therefore improve patient compliance with follow up screening.

Any method of detecting, measuring or quantitating the amount of metabolite(s) in a biological sample can be used; whether the metabolites are assayed individually, in combination, or by high-throughput methods. Preferred methods are reliable, sensitive and specific for a particular metabolite used as a biomarker in aspects of the present invention. The skilled artisan will recognize which detection methods are appropriate based on the sensitivity of the detection method and the abundance of the target metabolite. Depending on the sensitivity of the detection method and the abundance of the target metabolite, amplification may or may not be required prior to detection. One skilled in the art will recognize the detection methods where metabolite amplification is preferred.

The metabolite biomarkers of the present invention can be can be detected with standard technology, for example, mass spectrometry (MS) is widely used for routine population-based screening of all (e.g., screening newborns for metabolic disorders). This process has been the practice for 50 years; it is robust and has a short turn-around time for clinical use.

The metabolites detected in the inventive method can also be measured in functional body tissue and organs using magnetic resonance imaging (MRI). Magnetic Resonance Spectroscopy (MRS) is an MRI technique that can measure metabolite concentrations in living tissue. This technique could be used to non-invasively distinguish cancer from normal tissue and detect cancer recurrence or spread to different tissues e.g. extra pelvic or lymph node based on the concentration of distinguishing metabolites. Proton magnetic resonance spectroscopy (1H-MRS) is one form of MRS. MRI imaging can be used for the detection and measurement of (e.g., concentration of) metabolite levels not only in tissues, but also or in fluids.

The levels of the metabolite biomarkers of the present invention also can be detected with one or more of the following devices/methods for detecting metabolites: NMR, mass spectrometry (MS), MRI, gas chromatography (GC), High performance liquid chromatography (HPLC), capillary electrophoresis (CE), desorption electrospray ionization (DESI), laser ablation ESI (LAESI), ion-mobility spectrometry, electrochemical detection (coupled to HPLC), and Raman spectroscopy and radiolabel (when combined with thin-layer chromatography).

Any assay that will detect the metabolite biomarkers of the present invention can be used. Another example is a device for detecting and measuring metabolite levels called the “iknife,” which was developed at Imperial College, London, England. (Júlia Balog, László Sasi-Szabó, James Kinross, et al. Intraoperative Tissue Identification Using Rapid Evaporative Ionization Mass Spectrometry. Science Translational Medicine, 2013; 5 (194). This device captures smoke from electrosurgical cauterization of tissues at surgery and, using metabolomics (mass spectrometry) analysis of the metabolites in the smoke, is able to distinguish cancer from normal tissue. The metabolite biomarkers of the present invention could be measured at the time of surgery to assess surgical margins to ensure that tissue left behind do not contain cancer cells i.e. surgical margins are clear. Further, using this technique can be used to provide real time evaluation of cancer spread to various parts of the uterus e.g. the cervix, pelvis, and extra-pelvic areas. In particular this could also provide real-time intra-operative assessment of spread to the various lymphatic node groups and to other sites in the abdomen and pelvis surgical. Other methods for metabolite detection include the collection of hair or nail samples that can be appropriately prepared using existing methods for Mass Spectrometry analysis and the use of a Q-tip swab to collect fluid from the uterine cavity that collects in the posterior fornix of the vagina (swabbing will absorb the fluid). The fluid will then be leached into a standardized buffer by placing the used swab into the fluid. The specimens can then be tested using NMR or MS or other methods currently in use. Further descriptions of detection methods are described above and also below in the Examples.

An advantage of the present diagnostic invention is that it is a rapid, relatively inexpensive and non-invasive method for diagnosing and assessing the prognosis of individuals to develop or be at risk for endometrial cancer, to have asymptomatic or early-stage endometrial cancer, or to be symptomatic of endometrial cancer. In some aspects, tests may be performed multiple times on the same subject to assess disease progress. One embodiment of the present inventive method comprises assaying a patient biological sample for a level of a specific metabolite(s) in the biological sample, wherein a different level of the specific metabolite(s) in the biological sample as compared to a statistically validated threshold for each specific metabolite(s) indicates endometrial cancer in the patient.

In certain aspects of the present invention, and as otherwise described herein, metabolite detection includes detecting the level of (e.g., the concentration of) one or more of the metabolites in the biological sample. The one or more metabolites in the biological sample may be at a different level than a statistically validated threshold for the one or more metabolites. The statistically validated threshold for the level of the specific metabolite(s) is based upon the level of each specific metabolite(s) in comparable control biological samples from a control population, e.g., from subjects that do not have endometrial cancer. Various control populations are otherwise described herein. The statistically validated thresholds are related to the values used to characterize the level of the specific metabolite(s) in the biological sample obtained from the subject or patient. Thus, if the level of the metabolite is an absolute value, then the control value is also based upon an absolute value.

The statistically validated thresholds can take a variety of forms. For example, a statistically validated threshold can be a single cut-off value, such as a median or mean. Or, a statistically validated threshold can be divided equally (or unequally) into groups, such as low, medium, and high groups, the low group being individuals least likely to have endometrial cancer and the high group being individuals most likely to have endometrial cancer.

Statistically validated thresholds, e.g., mean levels, median levels, or “cut-off” levels, may be established by assaying a large sample of individuals in the select population and using a statistical model such as the predictive value method for selecting a positivity criterion or receiver operator characteristic curve that defines optimum specificity (highest true negative rate) and sensitivity (highest true positive rate). A “cutoff value” may be separately determined for the level of each specific metabolite assayed. Statistically validated thresholds also may be determined according to the methods described in the Examples hereinbelow.

The levels of the assayed metabolites in the patient biological sample may be compared to single control values or to ranges of control values. In one embodiment, a specific metabolite in a biological sample from a patient (e.g., a patient having or suspected of having endometrial cancer) is present at an elevated or reduced level (i.e., at a different level) than the specific metabolite in comparable control biological samples from subjects that do not have endometrial cancer when the level of the specific metabolite in the patient biological sample exceeds a threshold of one and one-half standard deviations above the mean of the concentration as compared to the comparable control biological samples. More preferably, a specific metabolite in a biological sample from a patient (e.g., a patient having or suspected of having endometrial cancer) is present at an elevated or reduced level (i.e., at a different level) than the specific metabolite in comparable control biological samples from subjects that do not have endometrial cancer when the level of the specific metabolite in the patient biological sample exceeds a threshold of two standard deviations above the mean of the concentration as compared to the comparable control biological samples. In another embodiment, a specific metabolite in a biological sample from a patient (e.g., a patient having or suspected of having endometrial cancer) is present at an elevated or reduced level (i.e., at a different level) than the specific metabolite in comparable control biological samples from subjects that do not have endometrial cancer when the level of the specific metabolite in the patient biological sample exceeds a threshold of three standard deviations above the mean of the concentration as compared to the comparable control biological samples.

If the level of a specific metabolite/metabolites in the patient biological sample is present at different levels than its/their respective statistically validated threshold(s), then the patient is more likely to have endometrial cancer than are individuals with levels comparable to the statistically validated threshold(s). The extent of the difference between the subject's levels and statistically validated thresholds is also useful for characterizing the extent of the risk and thereby, determining which individuals would most greatly benefit from certain therapies, e.g., aggressive therapies. In those cases, where the statistically validated threshold ranges are divided into a plurality of groups, such as statistically validated threshold ranges for individuals at high risk of endometrial cancer, average risk of endometrial cancer, and low risk of endometrial cancer, the comparison involves determining into which group the subject's level of the relevant risk predictor falls.

A “reduced level” or an “elevated level” of a metabolite refer to the amount of expression or concentration of a metabolite in a biological sample from a patient compared to statistically validated thresholds, e.g., the amount of the metabolite in biological sample(s) from individual(s) that do not have endometrial cancer, have endometrial cancer (or a particular severity or stage of endometrial cancer), or have other reference diseases. For example, a metabolite has a “reduced level” in the serum from a subject when the metabolite is present at a lower concentration in the subject's serum sample than in serum from a subject who does not have endometrial cancer; and a metabolite has an “elevated level” in the serum from a subject when the metabolite is present at a higher concentration in the subject's serum sample than in serum from a subject who does not have endometrial cancer. For certain metabolites, elevated levels in a biological sample indicate the presence of or a risk for endometrial cancer; at the same time, other metabolites may be present in reduced levels in patients or subjects with endometrial cancer. In either of these example situations, metabolites are at a “different level” in endometrial cancer subjects versus healthy controls.

The differential expression of a particular biomarker indicating a diagnosis or prognosis for endometrial cancer may be more than, e.g., 1,000,000×, 100,000×, 10,000×, 1000×, 10×, 5×, 2×, 1× a particular statistically validated threshold, or less than, e.g., 0.5×, 0.1×, 0.01×, 0.001×, 0.0001×, 0.000001× a particular statistically validated threshold.

The metabolite biomarker methods of the present invention also can be combined with non-biomarker-based diagnostics (performed before, after, or concurrently) to improve endometrial cancer diagnosis and for continued monitoring of the effect of treatment and/or the disease process. In one embodiment, a diagnosis of endometrial cancer using the present metabolite biomarker methods can be confirmed with or validated by structural information about the patient. For example, a trans-vaginal ultrasound of the uterus to determine the thickness of endometrium can be performed either before or after determining the level of one or more, or a combination of, the metabolite biomarkers of the present invention. Further, imaging techniques, such as MRI, Ultrasound or CT, also can be used to detect spread of the cancer from deeper penetration of the uterine muscle to more distant sites. Deeper invasion of endometrial cancer into the uterine muscle increases the risk of distant spread that might not be apparent on physical exam. The extent and likelihood of spread could be further assessed while in the operating room by opening up the uterus that has been removed and examining it. In addition, rapid histologic exam also called “frozen section” also could performed while the surgeon is still in the operating room to evaluate for possible cancer spread to nodes.

The metabolite biomarker methods of the present invention also can be combined with a physical pelvic exam to assess the size of the uterus and to search for evidence of spread of the cancer from the body of the uterus to other anatomical areas. Spread beyond the uterine body changes both the prognosis and the therapy that is required. Areas of spread include, e.g. the cervix which is the lowest aspect of the uterus and the extra-uterine pelvis. Tumor extension to the superficial lymph nodes (inguinal, pelvic, abdominal or more distant sites such as the superclavicular). The presence of masses in the abdomen or any other sites, or the presence of ascites, provide preoperative evidence of distant spread of the cancer from the uterus to the abdomen. Physical exam, while necessary, has significant limitations and cannot be relied on solely to determine the extent of cancer spread. Thus, it would be beneficial to combine physical exam with the metabolite biomarker methods of the present invention.

In another embodiment, the present metabolite biomarker methods can be combined with one or more other non-biomarker-based diagnostics of endometrial cancer, such as, evaluation of abnormal vaginal bleeding, endometrial biopsy, dilation and curettage, and/or risk profile evaluation (for example, histological tumor grading and depth of tumor invasion; early menarche/late menopause; family history of cancer or a cancer syndromes e.g. 0 syndrome and Lynch syndrome; family history of ovarian, breast, endometrial or colon cancer; women 50-70 years old; hormone therapy, e.g., estrogen therapy; estrogen secreting tumors; post-menopausal women with vaginal bleeding; fatty diet; obesity, a very significant risk factor and one that currently is present in epidemic numbers of American women; polycystic ovary disease; tamoxifen therapy; those with precancerous dysplastic changes of the endometrium, such as endometrial hyperplasia; diabetes mellitus; hypertension; age; race; and/or smoking).

Also, the present metabolite biomarker methods can be combined with other biomarker-based diagnostics; examples would include, but are not limited to, serum protein biomarkers CA125, HE4, and growth differentiating factor (GDF).

More or less aggressive treatment can be administered to the patient depending on whether diagnosis using the present biomarker methods is confirmed by one or more of the alternative method of diagnosis.

Beyond disease prediction, the present metabolite biomarker methods also can be combined with treating endometrial cancer in a subject. In addition to the detection and diagnostic methods described above, the inventive methods also can include, administering a therapeutically effective amount of a treatment for endometrial cancer to the diagnosed subject. That is, the present metabolite biomarker methods can be combined with the treatment of endometrial cancer, i.e., to indicate the initiation of one or more endometrial cancer therapies, discontinuation of one or more therapies, or an adjustment to one or more therapies (e.g., an increase or decrease to chemotherapy or drug therapy). The present metabolite biomarker methods also will allow for early prediction of endometrial cancer, treatment at an early stage of the cancer, and for targeted therapy to reduce the likelihood or prevent the disease from progressing to a later stage endometrial cancer. In response to the diagnosis of endometrial cancer, in some aspects of the method, a subject may be treated with one or more of endometrial cancer treatments (e.g., a surgery, radiation therapy, chemotherapy, and/or a drug,), or treated with a modification of an existing treatment, modified in response to the diagnosis or prognosis of endometrial cancer in that subject.

In response to the diagnosis of endometrial cancer based on the present metabolite biomarker methods, additional therapeutic measures beyond surgery may be needed to control cancer reoccurrence or to eliminate cancer cells that have spread microscopically. These additional therapeutic measures are called “adjuvant” therapy. The primary such adjuvant agent is radiation therapy (RT). RT is particularly indicated if patient is at high risk for local recurrence or the cancer may have or is likely to have spread further beyond the uterus. Chemotherapy is less commonly used. The combined use of adjuvant RT and chemotherapy particularly in more advanced disease, called “combined adjuvant therapy,” can potentially reduce the risk of local recurrence of cancer in the pelvis and distant metastasis.

Another aspect of the invention is a method of providing medical services for a patient suspected of having or having endometrial cancer, including a physician, or other individual requesting a biological sample from and diagnostic information about the patient, wherein the diagnostic information is a level of one or more metabolites C14.2, PC ae C38:1, 3-Hydroxybutyric acid, C18:2, PC ae C40:1, and C6 (C4:1-DC) in the biological sample; and the physician, or other individual administering a therapeutically effective amount of a treatment for endometrial cancer when the diagnostic information indicates that the level of the one or more metabolites in the biological sample is at a different level than a statistically validated threshold for the one or more metabolites.

In some embodiments, the metabolite biomarker methods of the present invention can be combined with, and/or used for the selection of, various treatments for endometrial cancer. Different treatments for endometrial cancer can be ordered by or administered by a physician, or other healthcare provider, for a patient depending on the stage or severity of the endometrial cancer (early or late stage; or FICO stages I-IV) as indicated by the metabolite biomarker methods of the present invention. For example, if there is a statistically significant difference between only one or two of the present biomarkers and their controls, then the endometrial cancer might be considered to be early stage, and then only surgery (e.g., a hysterectomy) is recommended, ordered or performed by the physician, or other healthcare provider. Standard surgery is a total abdominal hysterectomy and bilateral salpingo-oophorectomy (i.e. removal of the fallopian tubes and both ovaries. Surgery is usually curative for patients with no evidence of or at low risk of spread EC spread. Further, chemotherapy or radiation therapy might be recommended, ordered or performed by the physician, or other healthcare provider, if there is a statistically significant difference three or more of the present biomarkers and their controls.

Further, the aggressiveness of the treatment could be based on the degree or amount of difference between the levels of the present biomarkers and their controls. For example, a specific metabolite in a biological sample from a patient may be present at an elevated or reduced level as compared to comparable control biological samples and the level of the specific metabolite in the biological sample exceeds a threshold of three standard deviations above the mean of the concentration as compared to the control biological samples; in which case more aggressive treatment (e.g., chemotherapy or radiation therapy) might be recommended, ordered or performed by the physician, or other healthcare provider

In some embodiments, the treatment is administered in a therapeutically effective amount. The therapeutically effective amount will vary depending upon a variety of factors including, but not limited to: the stage or severity of the endometrial cancer (early or late stage; or FICO stages I-IV) as indicated by the metabolite biomarker methods; the metabolite levels; the age, body weight, general health, sex, and diet of the subject; the rate of excretion of any drug; any drug combination; and the mode and time of administration of the treatment.

One treatment for endometrial cancer is for a physician, healthcare provider, or other individual to advise (or order) the patient to have surgery. However, if the present metabolite biomarker methods indicate late-stage endometrial cancer, a patient might have radiation therapy. Also, if the present metabolite biomarker methods indicate late-stage severe endometrial cancer, the physician, or other healthcare provider, could order chemotherapy.

The metabolite biomarker methods of the present invention also can be combined with, and/or used for the selection and administration of, various medications for the treatment of endometrial cancer. By using the present metabolite biomarker methods, a physician or other healthcare provide can determine whether medication is needed and, if so, the amount and type of the medication to be administered. Examples of endometrial cancer medications include, but are not limited to, cyclophosphamide, doxorubicin, cisplatinum, medroxy-progesterone acetate and other progestational agents. If the levels of the present biomarkers indicate that the endometrial cancer is early-stage, treatment for the patient then might exclude toxic therapeutic or chemotherapeutic agents.

Accurate prognostication is an important objective in endometrial cancer patient management. Accurate prognostication would be very beneficial in helping to assure appropriate patient and family counseling and to assess the likelihood of significant adverse outcomes including death and severe morbidities among survivors. In some aspects, the metabolite level is used to determine the efficacy of treatment received by a patient for endometrial cancer (e.g., surgical removal, RT, or chemotherapy). That is, the metabolite levels of the patient may be assessed before treatment, and on one or more occasions after the administration of a treatment, to determine whether the treatment is effective. In particular, the present methods for diagnosing and treating also include performing the present metabolite biomarker methods on multiple occasions, i.e., to monitor the treatment effect and/or the brain condition of the patient over time. In particular, at one or more moments in time after initially performing the present metabolite biomarker methods, the present methods can again be performed and the results compared to results from an earlier-performed use of the present metabolite biomarker methods. A treatment for endometrial cancer can be administered before or after initially performing the present metabolite biomarker methods; and the course of treatment can be altered as indicated by the comparison(s). For example, if a endometrial cancer medication has been administered and, with the passage of time, there is a greater difference between the amount of a biomarker and its control, then a larger dose of the medicament might be indicated.

In addition, the metabolite biomarker methods of the present invention can be can be used as short and long-term evidence of disease recurrence after treatment, whether locally in the pelvis or through more distant metastasis to the abdomen and beyond. The metabolomics profile would be expected to shift if there is a reoccurrence of cancer. These metabolomics changes would be expected to manifest in any body fluid sampled e.g. vaginal swabs, blood, saliva, sweat, breath condensate, urine or on analyses of hair or nail samples. Thus, the need for frequent post-treatment surveillance visits to the doctor's office could be minimized while the frequency of actual surveillance could be increased including the use of serial metabolite measurements to identify early recurrence of the cancer.

One embodiment of the present inventive method is the monitoring of treatment for endometrial cancer in a human patient, comprising: requesting a first biological sample from and first diagnostic information about the patient, wherein the first diagnostic information is a level of one or more metabolites C14.2, PC ae C38:1, 3-Hydroxybutyric acid, C18:2, PC ae C40:1, and C6 (C4:1-DC) in the first biological sample; administering a therapeutically effective amount of a treatment for endometrial cancer to the patient; after administering the therapeutically effective amount of the treatment for endometrial cancer to the patient, requesting a second biological sample from and second diagnostic information about the patient, wherein the second diagnostic information is a level of one or more metabolites C14.2, PC ae C38:1, 3-Hydroxybutyric acid, C18:2, PC ae C40:1, and C6 (C4:1-DC) in the second biological sample; and comparing the first diagnostic information and the second diagnostic information to determine whether the level of the one or more metabolites in the first biological sample is at a different level than the level of the one or more metabolites in the second biological sample.

Kits

Another embodiment of the present invention is a kit for diagnosing endometrial cancer. Kits that allow for the targeted measure of one or more metabolites would reduce both overall cost and turn-around time for a diagnosis of endometrial cancer.

In one embodiment, a biomarker panel is used to diagnose endometrial cancer. The panel would be configured to detect two or more of C14.2, PC ae C38:1, 3-Hydroxybutyric acid, C18:2, PC ae C40:1, and C6 (C4:1-DC). For example, an MS or NMR based targeted kit (i.e. focusing on a limited number of metabolites, rather than the entire >300 metabolites analyzed. This would reduce cost and turn-over time.

In one embodiment, the present diagnostic methods and kits are useful for determining if and when medical treatments and therapeutic agents that are targeted at treating endometrial cancer should or should not be prescribed for an individual patient. Such medical treatments and therapeutic agents are discussed above and/or are known in the art, and will be ordered by or prescribed by a physician (or other healthcare provider) based on results of the inventive method and standard medical practices.

### EXAMPLES

Without further elaboration, it is believed that one skilled in the art can, using the preceding description, practice the present invention to its fullest extent. The following detailed examples describe how to perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Materials and Methods

Preoperative venous blood was collected from women diagnosed with EC at the Roswell Park Cancer Institute (Buffalo, N.Y.). The serum was stored at −80° C. and was not thawed until metabolomic analysis. All patients signed a written consent. The study protocol was approved by the IRB at RPCI. Specimens were collected as part of a biobanking project in which tissue and blood specimens of cancer patients are archived for future scientific study. Control specimens were obtained and archived from women without EC or other neoplastic disorders. Patient demographic and clinical information including age, race (Caucasian and African American), BMI, history of diabetes mellitus, use of hormonal replacement therapy and tamoxifen use were obtained. Disease staging based on the FIGO classification system was ascertained. For the sake of uniformity, only the endometriod histological type of EC was used. Further, only cases with no prior diagnosis or treatment of any cancer namely surgery, radiation or chemotherapy were included. Similar criteria were used for controls namely no history of other cancers or radiation or chemotherapy for any reason. A total of 46 early-stage EC cases (FIGO stages I-II) that did not extend beyond the uterus and 10 cases (FIGO stages III-IV) in which the disease extended beyond the uterus constituted the study group. A total of 60 unaffected control samples were used in the study.

Both Direct Injection-Mass Spectrometry (DI-MS) and NMR based metabolomic analysis was performed.

NMR Metabolomic Analysis

The inventors previously extensively described the techniques for NMR (Bahado-Singh R O, Akolekar R, Mandal R, Dong E, Xia J, Kruger M, et al. First-trimester metabolomic prediction of late-onset preeclampsia. Am J Obstet Gynecol; 2013:208: 58.e1-7; Bahado-Singh R O, Akolekar R, Mandal R, Dong E, Xia J, Kruger M et al. Metabolomics and first-trimester prediction of early-onset preeclampsia. J Mat Fet Neonat Med 2012; 25:1840-7). To summarize, the Vernon Inova 500 MHz NMR spectrometer was used (International Equipment Treating limited, Vernon Hills, Ill.). Serum samples were filtered through 3-kd cut off centrifuge filter units (Amicon Micron YM-3; Sigma-Aldrich, St. Louis, Mo.) to remove blood proteins. Three hundred and fifty microliters of samples was put into the centrifuge filter device and spun (10,000 rpm for 20 minutes) so as to remove macro molecules including proteins and lipoproteins. If the total volume of sample was <300 μl a 50-mmol NaH2PO4 buffer (pH7) was added to reach a total volume of sample 300 Metabolite concentrations were adjusted for the dilution due to the buffer. Thereafter, 35 μl of D2O and 15 μl of buffer solution (11.667 mmol disodium-2, 2-dimethyl-2-silceptentane-5-sulphonate, 730 mmol imidazole and 0.47% NaN3 in H2O) was added to the sample.

A total of 350 μl of sample was transferred to a micro cell NMR tube (Shigemi, Inc., Allison Park, Pa.). 1H-NMR spectra were collected on a 500-MHz Inova (Varian Inc, Palo Alto, Calif.) spectrometer with a 5-mm ITCN Z-gradient PFG cold-probe. The singlet produced by the disodium-2,2-dimethyl-2-silcepentane-5-sulphonate methyl groups was used as an internal standard by which to measure the chemical shift. The standard reference substance was set at 0 ppm and used for quantification of metabolites of interest. The 1H-NMR spectra were analyzed with a Chenomx NMR Suite Professional Software package (Version 7.1:Chenomx Inc. Edmonton, Alberta, Canada). This permits quantitative and qualitative analysis of the NMR spectrum observed the NMR spectrum was manually fitted to an internal database to the observed spectrum. Each spectrum was evaluated by at least 2 NMR spectroscopists to minimize errors of quantitation and identification.

Combined Direct Injection (DI) and LC-MS/MS Compound Identification and Quantification

Targeted quantitative metabolomics analysis of the serum was performed by combining direct injection mass spectrometry (AbsoluteIDQ™ Kit) with a reverse-phase LC-MS/MS Kit. The Kit is a commercially available from BIOCRATES Life Sciences AG (Austria). In combination with an ABI 4000 Q-Trap (Applied Biosystems/MDS Sciex) mass spectrometer targeted identification and quantification of up to 180 different endogenous metabolites including amino acids, acylcarnitines, biogenic amines, glycerophospholipids, sphingolipids and sugars. Derivatization and extraction of analytes, and the selective mass-spectrometric detection using multiple reaction monitoring (MRM) pairs was performed. Isotope-labeled internal standards and other internal standards are integrated in Kit plate filter for metabolite quantification.

The AbsoluteIDQ kit contains a 96 deep-well plate with a filter plate attached with sealing tape, and reagents and solvents used to prepare the plate assay. Of the first 14 wells in the Kit were used as follows: one for the blank, three zero samples, seven standards and three quality control samples provided with each Kit. All the serum samples were analyzed with the AbsoluteIDQ kit as described in the AbsoluteIDQ user manual. Serum samples were thawed on ice and were vortexed and centrifuged at 13,000×g. Ten μL of each serum sample was loaded onto the center of the filter on the upper 96-well kit plate and dried in a nitrogen stream. Subsequently, 20 μL of a 5% solution of phenyl-isothiocyanate was added for derivatization. After incubation, the filter spots were dried again using an evaporator. Extraction of the metabolites was then achieved by adding 300 μL methanol containing 5 mM ammonium acetate.

The extracts were obtained by centrifugation into the lower 96-deep well plate, followed by a dilution step with kit MS running solvent. Mass spectrometric analysis was performed on an API4000 Qtrap® tandem mass spectrometry instrument (Applied Biosystems/MDS Analytical Technologies, Foster City, Calif.) equipped with a solvent delivery system. The samples were delivered to the mass spectrometer by a LC method followed by a direct injection (DI) method. The Biocrates MetIQ software was used to control the entire assay workflow, from sample registration to automated calculation of metabolite concentrations to the export of data into other data analysis programs. A targeted profiling scheme was used to quantitatively screen for known small molecule metabolites using multiple reaction monitoring, neutral loss and precursor ion scans. The above description represents a summation of the inventors' previously published description of the methods.

Statistical Analysis

The metabolomic data was normalized using log scaling. Metabolomics involves the simultaneous analysis of a large number of metabolites. In this case, DI-MS measured 149 metabolites and NMR measured 32 metabolites. Principal Component Analysis (PCA) was used to achieve dimensional reduction and thus prioritize metabolites based on their contribution (Wishart D S. Computational approaches to metabolomics. Methods Mol Biol; 593:283-313). The separation of EC cases and controls achieved by the principal components (metabolites) were represented on cluster plots. Partial Least Squares Discriminant Analysis (PLS-DA) was utilized to optimize the separation between cases and controls. This involved rotating different combination of metabolites to identify the principal components which achieved maximum separation or discrimination between cases and controls (Xia J, Mandal R, Sineinkov I V, Broadhurst D, Wishart D. MetaboAnalyst 2.0: a comprehensive server for metabolomics data analysis. Nucleic Acid Res 2012; 40:W127-23). A total of 2000 rounds of permutation testing were performed to determine whether the observed separation or discrimination between EC and control cases was due to chance. The MetaboAnalyst computer program (Xia J, Psycliogious N, Young N, Wishart D E. MetaboAnalyst: a web server for metabolomic data analysis and interpretation, Nucleic Acid Res 2009; 37:W652-W660) was used to perform PCA, PLS-DA and permutation testing. In addition, a Variable Importance Plot (VIP) was used to rank metabolites based on their importance in EC identification. In a VIP plot the higher the value on the x-axis for a particular metabolite, the greater is its relative value for distinguishing cases from controls. The statistical approach described has been extensively reported by us (Bahado-Singh R O, Akolekar R, Mandal R, Dong E, Xia J, Kruger M, et al. First-trimester metabolomic prediction of late-onset preeclampsia. Am J Obstet Gynecol; 2013:208: 58.e1-7; Bahado-Singh R O, Akolekar R, Mandal R, Dong E, Xia J, Kruger M et al. Metabolomics and first-trimester prediction of early-onset preeclampsia. J Mat Fet Neonat Med 2012; 25:1840-7) previously.

The inventors used a cross validation (CV) technique to develop a biomarker model for the detection of EC and to validate this model in independent subgroups of cases and controls (Xia J, Broadhurst D I, Wilson M, Wishart D A. Translational biomarker discovery in clinical metabolomics: an introductory tutorial. Metabolomics 2013; 9:280-99). In k-fold CV, the entire patient group is divided into K subsets of equal size. Of these K subsets, one subset is used for validation of the model that was generated by the remaining (K−1) subsets.

To perform independent validation of the model the entire data-set was randomly divided into a group used to develop the predictive algorithm “training set” and an independent validation group or “test-set” in which the algorithm performance was evaluated. The groups were split as follows: 60% of all cases (EC and controls) were randomly assigned to the “training set” and 40% to the “test-set”. Allocation was such that there were no significant differences e.g. demographic and potentially confounding variables between the two groups. Model optimization in the training group was achieved using the cross-validation (CV) technique. Ten rounds of CV were performed and the final model is the one with the optimal diagnostic effectiveness. The diagnostic accuracy of the model was then tested in the independent validation group, which as pointed out previously consisted of cases and controls that were not used in deriving the model.

The (LASSO) Least Absolute Shrinkage and Selection Operator technique (Tibshirani R. Regression shrinkage and selection via LASSO., J.R. Statist Soc 1996; 58:267-88.) using 10-fold cross-validation was used for variable selection in the regression. Stepwise variable selection (Hastie T, Tibshirani R, Friedman J. The elements of statistical learning: Data mining, Inference, and Prediction. 2nd Edition. Springer Series in Statistics. Springer-Verlag, 2009. Springer, NY.) using 10-fold cross-validation was used to optimize the logistic regression model.

The area under the Receiver Operating Characteristics Curve (AUROC or AUC) (Xia J, Broadhurst D I, Wilson M, Wishart D S. Translational biomarker discovery in clinical metabolomics: an introductory tutorial. Metabolomics 2013; 9:280-99) were calculated to compare the performance of each model. The free MetaboAnalyst web server (Xia J, Psychogios N, Young N, Wishart DS. MetaboAnalyst: a web server for metabolomic data analysis and interpretation. Nucleic Acid Res 2009; 37:W652-W660) was used to perform PCA, PLS-DA and permutation analyses. Custom programs written using the R statistical software package and the STATA 12.0 programs were uses for all other statistical analyses. Details of these statistical analyses were reported in a previous publication by the inventors (ref. Bahado-Singh R O et al. Early PE validation study-accepted for publication Am J Obstet Gynecol 2015). T-test, Fisher's Exact and Pearson Chi-Square were used for comparisons. Bonferroni correction for multiple testing was performed.

### Example 2

### Results

The inventors tested a total of 116 specimens, 56 with EC and 60 normal controls. A total of 181 metabolites, 149 based on DI-MS and 32 based on NMR were analyzed. This was after excluding metabolites with the percent of missing values in more than 20% of cases for both patient groups. Table 1 shows the demographic and clinical comparisons of EC and controls.

TABLE 1
Endometrial Cancer versus Controls:
Demographic and Clinical Variables

| | Endometrial | | Normal | | p |
|---|---|---|---|---|---|
| Parameter | Cancer | | Controls | | value |
| Number of cases | 56 | | 60 | | |
| Age, years, mean (SD) | 59.1 | (12.8) | 59.2 | (12.7) | 0.737^{#} |
| Racial origin, n (%) | | | | | |
| White | 52 | (92.9) | 56 | (93.3) | 1.000⁺ |
| Black | 4 | (7.1) | 4 | (6.7) | |
| Diabetes, n (%) | 13 | (23.2) | 2 | (3.3) | 0.002⁺ |
| BMI, mean (SD) | 36.9 | (17.3) | 28.8 | (6.8) | 0.002^{#} |
| HRT, n (%) | 13 | (23.2) | 20 | (33.3) | 0.227⁺⁺ |
| Tamoxifen, n (%) | 1 | (1.8) | 3 | (5.0) | 0.619⁺ |

Table 1:
^{#}t-test,
⁺Fisher's Exact,
⁺⁺Pearson Chi-square

Unsurprisingly, there was a significantly higher frequency of diabetics and a higher mean body mass index (BMI) in EC compared to control cases. Spearman correlation analysis between BMI and diabetes was performed and the two variables were highly correlated, Spearman's rho=0.358, p=0.001. The FIGO staging of the EC cases is listed in Table 2.

TABLE 2
FIGO stage of Endometrial Cancer cases

| FIGO Stage | Number (%) EC cases | |
|---|---|---|
| Stage 0 | 60 | (51.7) |
| Stage I | 45 | (38.8) |
| Stage II | 1 | (0.9) |
| Stage III | 8 | (6.9) |
| Stage IV | 2 | (1.7) |

There were a total of 69 cases in the biomarker discovery subset and 47 in the validation subset. Table 3 compares the demographic and clinical characteristics of EC and controls for both the “discovery” and “validation” patient groups. Higher rates of diabetes and higher mean BMI were found in EC cases compared to controls in the discovery group while significantly higher rates of diabetes were seen for EC in the validation group compared to the controls.

TABLE 3
Demographic/Clinical Variables: EC and Controls in the Discovery and Validation Groups

| | Discovery Group | | | Validation Group | | |
|---|---|---|---|---|---|---|
| | Endometrial | | | Endometrial | | |
| | Cancer | Normal | p value | Cancer | Normal | p value |
| | Number of cases | | | | | |
| Parameter | 33 | 36 | — | 23 | 24 | — |
| Age, years, mean (SD) | 58.4 (13.8) | 59.7 (13.4) | 0.685 | 60.2 (11.5) | 58.5 (11.9) | 0.627^{#} |
| Racial origin, n (%) | | | 0.615 | | | 0.348⁺ |
| White | 32 (97.0) | 33 (91.7) | | 20 (87.0) | 23 (95.8) | |
| Black | 1 (3.0) | 3 (8.3) | | 3 (13.0) | 1 (4.2) | |
| Diabetes, n (%) | 8 (24.2) | 2 (5.6) | 0.040 | 5 (21.7) | 0 (0.0) | 0.022⁺ |
| BMI, mean (SD) | 39.1 (20.8) | 28.2 (6.8) | 0.007 | 33.5 (9.3) | 29.8 (6.6) | 0.126^{#} |
| HRT, n (%) | 10 (30.3) | 14 (38.9) | 0.613 | 3 (13.0) | 6 (25.0) | 0.461⁺ |
| Tamoxifen, n (%) | 1 (3.0) | 3 (8.3) | 0.615 | 0 (0.0) | 0 (0.0) | NA⁺ |

For Table 3: ^{#}t-test, ⁺⁻Fisher's Exact test

Table 4 compares the concentration of individual metabolites in the overall EC group compared to controls based on NMR analysis. The metabolite concentrations are expressed in μM/L. Of the 32 NMR based metabolites, a total of 4 metabolites were significantly altered in EC when controlled for multiple comparisons (q-value <0.05).

TABLE 4
NMR: Metabolite Concentrations EC vs Normal (All EC cases and controls)

| | Mean (SD) | | | Endometrial | | q- |
|---|---|---|---|---|---|---|
| | Endometrial | | | Cancer/ | Fold | value |
| | Cancer | Normal | p-value | Normal | Change | (FDR) |
| | Number of cases | | | | | |
| Metabolite | 56 | 60 | — | — | — | — |
| 1-Methylhistidine | 82.03 (82.56) | 83.05 (96.84) | 0.952 | Down | −1.01 | 0.989 |
| 2-Hydroxybutyrate | 46.50 (41.15) | 30.88 (26.35) | <0.001 | Up | 1.51 | 0.005 |
| Acetic acid | 42.91 (34.69) | 50.85 (46.36) | 0.296 | Down | −1.19 | 0.570 |
| Betaine | 65.92 (57.43) | 65.78 (33.03) | 0.988 | Up | 1.00 | 0.989 |
| L-Carnitine | 56.83 (44.17) | 74.70 (152.08) | 0.386 | Down | −1.31 | 0.654 |
| Creatine | 43.78 (37.65) | 52.04 (33.53) | 0.214 | Down | −1.19 | 0.456 |
| Citric acid | 173.24 (132.82) | 200.36 (389.48) | 0.612 | Down | −1.16 | 0.821 |
| Choline | 12.90 (10.89) | 11.57 (7.05) | 0.443 | Up | 1.11 | 0.698 |
| D-Glucose | 6470.54 (3474.65) | 5922.48 (3311.47) | 0.386 | Up | 1.09 | 0.654 |
| Glycine | 370.29 (266.25) | 419.42 (322.74) | 0.375 | Down | −1.13 | 0.654 |
| Glycerol | 496.93 (461.91) | 455.23 (440.22) | 0.620 | Up | 1.09 | 0.823 |
| Formic acid | 31.60 (19.42) | 31.96 (15.91) | 0.911 | Down | −1.01 | 0.959 |
| L-Glutamic acid | 65.24 (42.56) | 66.92 (47.98) | 0.843 | Down | −1.03 | 0.927 |
| L-Tyrosine | 82.93 (42.46) | 88.25 (50.12) | 0.540 | Down | −1.06 | 0.769 |
| L-Phenylalanine | 88.93 (49.04) | 86.54 (42.24) | 0.779 | Up | 1.03 | 0.917 |
| L-Alanine | 484.17 (270.31) | 495.44 (250.57) | 0.816 | Down | −1.02 | 0.927 |
| L-Proline | 180.94 (81.46) | 198.18 (102.44) | 0.320 | Down | −1.10 | 0.578 |
| L-Threonine | 129.20 (100.24) | 134.59 (99.67) | 0.772 | Down | −1.04 | 0.917 |
| L-Isoleucine | 48.08 (23.12) | 50.16 (33.19) | 0.695 | Down | −1.04 | 0.855 |
| L-Histidine | 60.54 (47.15) | 62.11 (29.59) | 0.832 | Down | −1.03 | 0.927 |
| Lysine | 176.35 (109.23) | 172.42 (112.84) | 0.850 | Up | 1.02 | 0.927 |
| L-Lactic acid | 1895.02 (1159.65) | 1793.69 (914.38) | 0.601 | Up | 1.06 | 0.818 |
| Pyruvic acid | 90.23 (47.43) | 83.53 (41.57) | 0.419 | Up | 1.08 | 0.672 |
| 3-Hydroxybutyric acid | 130.52 (158.80) | 47.23 (56.47) | <0.001 | Up | 2.76 | 0.000 |
| L-Arginine | 68.61 (38.72) | 75.72 (36.96) | 0.314 | Down | −1.10 | 0.578 |
| Creatinine | 89.51 (72.30) | 84.26 (54.20) | 0.661 | Up | 1.06 | 0.843 |
| L-Glutamine | 564.41 (227.03) | 556.75 (205.71) | 0.849 | Up | 1.01 | 0.927 |
| L-Leucine | 95.59 (45.75) | 99.02 (60.58) | 0.731 | Down | −1.04 | 0.882 |
| L-Methionine | 20.11 (10.68) | 27.07 (13.65) | 0.002 | Down | −1.35 | 0.015 |
| L-Valine | 179.96 (90.10) | 187.28 (113.03) | 0.702 | Down | −1.04 | 0.855 |
| Acetone | 28.16 (24.64) | 15.71 (10.41) | <0.001 | Up | 1.79 | 0.005 |
| Methanol | 365.14 (285.28) | 338.45 (253.62) | 0.595 | Up | 1.08 | 0.817 |

In Table 4, p-value is calculated with t-test; p-value with bold and underline is calculated by the Wilcoxon Mann Whitney test

For DI-MS based metabolomics analysis, a total of 53 of 149 metabolites were significantly altered in EC compared to controls (see, Table 5).

TABLE 5
Metabolite Concentrations: DI-MS-EC versus Controls (All cases and controls)

| | Mean (SD) | | | Endometrial | | q- |
|---|---|---|---|---|---|---|
| | Endometrial | | | Cancer/ | Fold | value |
| | Cancer | Normal | p-value | Normal | Change | (FDR) |
| | Number of cases | | | | | |
| Metabolite | 56 | 60 | — | — | — | — |
| C0 | 33.43 (7.92) | 36.17 (9.15) | 0.088 | Down | −1.08 | 0.235 |
| C10 | 0.28 (0.13) | 0.19 (0.11) | <0.001 | Up | 1.47 | 0.001 |
| C14:1 | 0.14 (0.05) | 0.11 (0.03) | <0.001 | Up | 1.3 | 0.004 |
| C14:2 | 0.05 (0.03) | 0.03 (0.02) | <0.001 | Up | 1.74 | 0.000 |
| C16 | 0.11 (0.03) | 0.09 (0.02) | <0.001 | Up | 1.27 | 0.000 |
| C18 | 0.05 (0.01) | 0.05 (0.01) | 0.819 | Down | −1.01 | 0.927 |
| C18:1 | 0.15 (0.04) | 0.11 (0.04) | <0.001 | Up | 1.3 | 0.001 |
| C18:2 | 0.07 (0.02) | 0.05 (0.02) | <0.001 | Up | 1.34 | 0.000 |
| C2 | 7.64 (3.38) | 5.68 (2.09) | 0.001 | Up | 1.34 | 0.010 |
| C3 | 0.28 (0.14) | 0.32 (0.12) | 0.009 | Down | −1.16 | 0.045 |
| C4 | 0.19 (0.10) | 0.20 (0.12) | 0.612 | Down | −1.06 | 0.821 |
| C6 (C4:1-DC) | 0.07 (0.03) | 0.05 (0.02) | <0.001 | Up | 1.52 | 0.000 |
| C5 | 0.13 (0.06) | 0.14 (0.06) | 0.268 | Down | −1.09 | 0.521 |
| C5-DC (C6-OH) | 0.02 (0.01) | 0.02 (0.01) | 0.006 | Up | 1.21 | 0.032 |
| C7-DC | 0.03 (0.01) | 0.02 (0.01) | <0.001 | Up | 1.33 | 0.003 |
| C8 | 0.19 (0.09) | 0.13 (0.07) | <0.001 | Up | 1.43 | 0.001 |
| lysoPC a C16:0 | 77.29 (15.68) | 83.29 (18.13) | 0.060 | Down | −1.08 | 0.181 |
| lysoPC a C16:1 | 2.64 (0.92) | 2.72 (0.90) | 0.646 | Down | −1.03 | 0.836 |
| lysoPC a C17:0 | 1.40 (0.41) | 1.65 (0.44) | 0.002 | Down | −1.18 | 0.015 |
| lysoPC a C18:0 | 24.54 (6.20) | 27.43 (7.10) | 0.016 | Down | −1.12 | 0.067 |
| lysoPC a C18:1 | 15.75 (4.54) | 18.49 (6.12) | 0.011 | Down | −1.17 | 0.052 |
| lysoPC a C18:2 | 25.77 (8.17) | 32.47 (15.93) | 0.006 | Down | −1.26 | 0.032 |
| lysoPC a C20:3 | 1.95 (0.70) | 1.89 (0.72) | 0.651 | Up | 1.03 | 0.836 |
| lysoPC a C20:4 | 6.23 (2.43) | 5.89 (1.81) | 0.404 | Up | 1.06 | 0.665 |
| lysoPC a C26:0 | 0.26 (0.11) | 0.23 (0.09) | 0.206 | Up | 1.1 | 0.452 |
| lysoPC a C26:1 | 0.16 (0.05) | 0.15 (0.04) | 0.101 | Up | 1.09 | 0.258 |
| lysoPC a C28:0 | 0.29 (0.08) | 0.28 (0.09) | 0.854 | Up | 1.01 | 0.927 |
| lysoPC a C28:1 | 0.47 (0.13) | 0.50 (0.13) | 0.209 | Down | −1.06 | 0.452 |
| PC aa C24:0 | 0.09 (0.04) | 0.09 (0.03) | 0.526 | Up | 1.04 | 0.758 |
| PC aa C28:1 | 3.15 (0.83) | 3.34 (0.77) | 0.188 | Down | −1.06 | 0.420 |
| PC aa C30:0 | 4.12 (1.26) | 4.42 (1.50) | 0.251 | Down | −1.07 | 0.504 |
| PC aa C30:2 | 0.67 (0.20) | 0.70 (0.20) | 0.519 | Down | −1.04 | 0.758 |
| PC aa C32:0 | 13.50 (3.07) | 13.38 (2.96) | 0.827 | Up | 1.01 | 0.927 |
| PC aa C32:1 | 16.30 (7.62) | 16.08 (8.56) | 0.883 | Up | 1.01 | 0.952 |
| PC aa C32:2 | 4.03 (1.35) | 4.34 (1.59) | 0.265 | Down | −1.08 | 0.521 |
| PC aa C32:3 | 0.83 (0.30) | 0.82 (0.30) | 0.909 | Up | 1.01 | 0.959 |
| PC aa C34:1 | 188.52 (46.07) | 197.49 (46.62) | 0.300 | Down | −1.05 | 0.570 |
| PC aa C34:2 | 370.97 (70.65) | 383.81 (68.36) | 0.322 | Down | −1.03 | 0.578 |
| PC aa C34:3 | 18.50 (5.17) | 19.87 (5.87) | 0.185 | Down | −1.07 | 0.419 |
| PC aa C34:4 | 1.93 (0.66) | 2.12 (0.72) | 0.152 | Down | −1.1 | 0.353 |
| PC aa C36:0 | 1.80 (0.53) | 2.17 (0.94) | 0.034 | Down | −1.21 | 0.124 |
| PC aa C36:1 | 50.95 (13.73) | 58.05 (15.48) | 0.016 | Down | −1.14 | 0.067 |
| PC aa C36:2 | 247.21 (59.60) | 268.28 (57.38) | 0.055 | Down | −1.09 | 0.179 |
| PC aa C36:3 | 144.57 (32.53) | 158.45 (38.24) | 0.013 | Down | −1.1 | 0.058 |
| PC aa C36:4 | 210.77 (46.59) | 211.03 (46.49) | 0.976 | Down | −1 | 0.989 |
| PC aa C36:5 | 20.22 (7.64) | 26.80 (14.93) | 0.020 | Down | −1.33 | 0.081 |
| PC aa C36:6 | 0.77 (0.27) | 1.01 (0.43) | 0.002 | Down | −1.31 | 0.019 |
| PC aa C38:0 | 2.60 (0.77) | 3.01 (0.99) | 0.032 | Down | −1.16 | 0.124 |
| PC aa C38:1 | 0.71 (0.28) | 0.79 (0.33) | 0.145 | Down | −1.12 | 0.341 |
| PC aa C38:3 | 54.98 (15.58) | 53.86 (16.10) | 0.704 | Up | 1.02 | 0.855 |
| PC aa C38:4 | 133.16 (31.13) | 131.83 (32.45) | 0.822 | Up | 1.01 | 0.927 |
| PC aa C38:5 | 63.13 (13.17) | 71.60 (16.91) | 0.005 | Down | −1.13 | 0.031 |
| PC aa C38:6 | 70.39 (22.83) | 79.78 (29.12) | 0.057 | Down | −1.13 | 0.180 |
| PC aa C40:2 | 0.24 (0.05) | 0.27 (0.08) | 0.023 | Down | −1.13 | 0.092 |
| PC aa C40:3 | 0.44 (0.09) | 0.48 (0.11) | 0.052 | Down | −1.09 | 0.173 |
| PC aa C40:4 | 4.15 (1.20) | 4.27 (1.46) | 0.652 | Down | −1.03 | 0.836 |
| PC aa C40:5 | 11.59 (3.38) | 12.16 (3.52) | 0.381 | Down | −1.05 | 0.654 |
| PC aa C40:6 | 27.19 (10.24) | 29.07 (11.27) | 0.350 | Down | −1.07 | 0.621 |
| PC aa C42:0 | 0.60 (0.18) | 0.61 (0.21) | 0.965 | Down | −1 | 0.989 |
| PC aa C42:1 | 0.31 (0.09) | 0.31 (0.10) | 0.984 | Down | −1 | 0.989 |
| PC aa C42:2 | 0.20 (0.04) | 0.23 (0.06) | 0.002 | Down | −1.15 | 0.015 |
| PC aa C42:4 | 0.18 (0.04) | 0.19 (0.04) | 0.083 | Down | −1.08 | 0.232 |
| PC aa C42:5 | 0.31 (0.09) | 0.34 (0.09) | 0.055 | Down | −1.1 | 0.179 |
| PC aa C42:6 | 0.42 (0.13) | 0.49 (0.11) | <0.001 | Down | −1.15 | 0.002 |
| PC ae C30:0 | 0.31 (0.09) | 0.33 (0.09) | 0.210 | Down | −1.07 | 0.452 |
| PC ae C30:1 | 0.14 (0.06) | 0.16 (0.06) | 0.238 | Down | −1.09 | 0.486 |
| PC ae C30:2 | 0.19 (0.03) | 0.19 (0.03) | 0.469 | Down | −1.02 | 0.719 |
| PC ae C32:1 | 2.33 (0.55) | 2.40 (0.52) | 0.528 | Down | −1.03 | 0.758 |
| PC ae C32:2 | 0.68 (0.16) | 0.71 (0.17) | 0.393 | Down | −1.04 | 0.659 |
| PC ae C34:0 | 1.32 (0.35) | 1.44 (0.34) | 0.033 | Down | −1.09 | 0.124 |
| PC ae C34:1 | 8.75 (2.04) | 9.39 (1.94) | 0.085 | Down | −1.07 | 0.232 |
| PC ae C34:2 | 10.21 (2.68) | 11.56 (2.75) | 0.010 | Down | −1.13 | 0.050 |
| PC ae C34:3 | 7.03 (2.20) | 8.21 (2.62) | 0.006 | Down | −1.17 | 0.032 |
| PC ae C36:0 | 0.58 (0.13) | 0.62 (0.15) | 0.094 | Down | −1.08 | 0.242 |
| PC ae C36:1 | 7.27 (1.76) | 8.19 (1.78) | 0.006 | Down | −1.13 | 0.032 |
| PC ae C36:2 | 13.92 (3.90) | 15.84 (3.99) | 0.004 | Down | −1.14 | 0.027 |
| PC ae C36:3 | 7.63 (1.89) | 8.79 (2.30) | 0.004 | Down | −1.15 | 0.027 |
| PC ae C36:4 | 17.31 (3.68) | 18.13 (4.10) | 0.261 | Down | −1.05 | 0.519 |
| PC ae C36:5 | 11.52 (2.61) | 12.55 (3.63) | 0.081 | Down | −1.09 | 0.228 |
| PC ae C38:0 | 1.83 (0.49) | 2.33 (0.85) | 0.001 | Down | −1.28 | 0.008 |
| PC ae C38:1 | 0.43 (0.17) | 0.53 (0.20) | 0.004 | Down | −1.24 | 0.027 |
| PC ae C38:2 | 1.96 (0.48) | 2.22 (0.59) | 0.015 | Down | −1.13 | 0.066 |
| PC ae C38:3 | 4.31 (0.89) | 4.45 (1.03) | 0.443 | Down | −1.03 | 0.698 |
| PC ae C38:4 | 15.28 (3.64) | 15.69 (3.74) | 0.547 | Down | −1.03 | 0.774 |
| PC ae C38:5 | 19.40 (3.93) | 20.94 (4.65) | 0.043 | Down | −1.08 | 0.152 |
| PC ae C38:6 | 7.61 (1.52) | 8.68 (2.27) | 0.012 | Down | −1.14 | 0.058 |
| PC ae C40:1 | 1.02 (0.22) | 1.28 (0.35) | <0.001 | Down | −1.26 | 0.001 |
| PC ae C40:2 | 1.61 (0.44) | 1.66 (0.43) | 0.581 | Down | −1.03 | 0.809 |
| PC ae C40:3 | 1.07 (0.25) | 1.07 (0.25) | 0.989 | Up | 1 | 0.989 |
| PC ae C40:4 | 2.42 (0.58) | 2.46 (0.64) | 0.752 | Down | −1.01 | 0.901 |
| PC ae C40:5 | 3.79 (0.87) | 4.00 (1.04) | 0.238 | Down | −1.06 | 0.486 |
| PC ae C40:6 | 4.35 (1.01) | 4.94 (1.38) | 0.027 | Down | −1.14 | 0.107 |
| PC ae C42:1 | 0.33 (0.06) | 0.37 (0.08) | 0.005 | Down | −1.12 | 0.032 |
| PC ae C42:2 | 0.48 (0.12) | 0.56 (0.15) | 0.001 | Down | −1.17 | 0.011 |
| PC ae C42:3 | 0.66 (0.18) | 0.73 (0.20) | 0.048 | Down | −1.11 | 0.168 |
| PC ae C42:4 | 0.93 (0.29) | 0.92 (0.25) | 0.966 | Up | 1 | 0.989 |
| PC ae C42:5 | 2.34 (0.59) | 2.30 (0.62) | 0.695 | Up | 1.02 | 0.855 |
| PC ae C44:3 | 0.10 (0.02) | 0.10 (0.02) | 0.127 | Down | −1.07 | 0.311 |
| PC ae C44:4 | 0.38 (0.12) | 0.37 (0.09) | 0.623 | Up | 1.03 | 0.823 |
| PC ae C44:5 | 1.88 (0.64) | 1.75 (0.54) | 0.238 | Up | 1.07 | 0.486 |
| PC ae C44:6 | 1.54 (0.52) | 1.46 (0.45) | 0.404 | Up | 1.05 | 0.665 |
| SM (OH) C14:1 | 7.24 (2.00) | 7.49 (1.71) | 0.476 | Down | −1.03 | 0.724 |
| SM (OH) C16:1 | 4.20 (1.27) | 4.16 (0.96) | 0.855 | Up | 1.01 | 0.927 |
| SM (OH) C22:1 | 17.78 (4.23) | 18.86 (4.50) | 0.185 | Down | −1.06 | 0.419 |
| SM (OH) C22:2 | 13.43 (3.12) | 14.50 (3.55) | 0.064 | Down | −1.08 | 0.189 |
| SM (OH) C24:1 | 1.61 (0.42) | 1.66 (0.42) | 0.485 | Down | −1.03 | 0.732 |
| SM C16:0 | 131.93 (28.15) | 133.88 (25.78) | 0.698 | Down | −1.01 | 0.855 |
| SM C16:1 | 22.95 (5.36) | 22.51 (4.63) | 0.640 | Up | 1.02 | 0.836 |
| SM C18:0 | 30.62 (9.21) | 27.73 (5.95) | 0.049 | Up | 1.1 | 0.168 |
| SM C18:1 | 17.28 (5.94) | 15.82 (4.13) | 0.129 | Up | 1.09 | 0.313 |
| SM C20:2 | 1.05 (0.40) | 0.99 (0.40) | 0.419 | Up | 1.06 | 0.672 |
| SM C22:3 | 3.05 (0.93) | 2.86 (1.15) | 0.312 | Up | 1.07 | 0.578 |
| SM C24:0 | 24.90 (5.49) | 26.65 (6.47) | 0.120 | Down | −1.07 | 0.297 |
| SM C24:1 | 51.57 (11.68) | 51.49 (12.95) | 0.971 | Up | 1 | 0.989 |
| SM C26:0 | 0.19 (0.06) | 0.20 (0.05) | 0.323 | Down | −1.05 | 0.578 |
| SM C26:1 | 0.40 (0.11) | 0.42 (0.13) | 0.466 | Down | −1.04 | 0.719 |
| Hexose | 5801.31 (1785.42) | 5300.06 (1293.27) | 0.088 | Up | 1.09 | 0.235 |
| Alanine | 404.09 (94.78) | 423.37 (104.95) | 0.303 | Down | −1.05 | 0.570 |
| Arginine | 104.48 (19.20) | 106.17 (25.84) | 0.690 | Down | −1.02 | 0.855 |
| Asparagine | 36.49 (9.45) | 42.56 (9.97) | 0.001 | Down | −1.17 | 0.009 |
| Aspartate | 15.94 (4.81) | 15.72 (6.72) | 0.840 | Up | 1.01 | 0.927 |
| Citrulline | 32.47 (12.22) | 36.67 (11.45) | 0.059 | Down | −1.13 | 0.180 |
| Glutamine | 712.00 (119.68) | 698.98 (118.19) | 0.557 | Up | 1.02 | 0.781 |
| Glutamate | 57.47 (22.80) | 45.85 (19.35) | 0.004 | Up | 1.25 | 0.027 |
| Glycine | 255.98 (96.13) | 285.57 (84.34) | 0.080 | Down | −1.12 | 0.228 |
| Histidine | 74.00 (13.12) | 80.92 (14.02) | 0.007 | Down | −1.09 | 0.037 |
| Isoleucine | 65.62 (18.93) | 65.22 (19.56) | 0.910 | Up | 1.01 | 0.959 |
| Leucine | 122.97 (35.11) | 124.80 (36.11) | 0.783 | Down | −1.01 | 0.917 |
| Lysine | 188.09 (39.82) | 187.88 (42.56) | 0.979 | Up | 1 | 0.989 |
| Methionine | 17.28 (2.89) | 19.87 (5.49) | 0.007 | Down | −1.15 | 0.036 |
| Ornithine | 68.98 (18.82) | 67.67 (18.30) | 0.703 | Up | 1.02 | 0.855 |
| Phenylalanine | 64.29 (12.04) | 66.03 (15.40) | 0.503 | Down | −1.03 | 0.752 |
| Proline | 195.27 (64.93) | 196.65 (59.18) | 0.905 | Down | −1.01 | 0.959 |
| Serine | 92.75 (22.59) | 101.04 (26.84) | 0.076 | Down | −1.09 | 0.221 |
| Threonine | 116.34 (27.43) | 121.83 (27.50) | 0.239 | Down | −1.05 | 0.486 |
| Tryotophan | 48.48 (11.66) | 51.95 (12.08) | 0.119 | Down | −1.07 | 0.297 |
| Tyrosine | 62.00 (15.58) | 66.74 (18.84) | 0.144 | Down | −1.08 | 0.341 |
| Valine | 196.30 (52.74) | 198.93 (51.02) | 0.785 | Down | −1.01 | 0.917 |
| Acetylornithine | 1.28 (0.62) | 1.36 (0.70) | 0.521 | Down | −1.06 | 0.758 |
| Asymmetric | 0.50 (0.10) | 0.46 (0.11) | 0.058 | Up | 1.09 | 0.180 |
| dimethylarginine | | | | | | |
| Symmetric | 0.58 (0.21) | 0.55 (0.16) | 0.460 | Up | 1.05 | 0.718 |
| dimethylarginine | | | | | | |
| Total dimethylarginine | 1.15 (0.38) | 1.11 (0.35) | 0.596 | Up | 1.03 | 0.817 |
| alpha-Aminoadipic | 76.26 (34.23) | 72.87 (20.44) | 0.523 | Up | 1.05 | 0.758 |
| acid | | | | | | |
| Creatinine | 2.07 (0.72) | 2.09 (0.60) | 0.846 | Down | −1.01 | 0.927 |
| Kynurenine | 0.65 (0.22) | 0.84 (0.34) | <0.001 | Down | −1.31 | 0.001 |
| Hydroxyproline | 8.56 (3.68) | 10.63 (6.73) | 0.043 | Down | −1.24 | 0.152 |
| Putrescine | 0.20 (0.13) | 0.16 (0.05) | 0.090 | Up | 1.2 | 0.237 |
| Serotonin | 0.49 (0.43) | 0.56 (0.38) | 0.383 | Down | −1.13 | 0.654 |
| Taurine | 103.76 (32.76) | 98.66 (34.11) | 0.416 | Up | 1.05 | 0.672 |
| 1-Methylhistidine | 82.03 (82.56) | 83.05 (96.84) | 0.952 | Down | −1.01 | 0.989 |
| 2-Hydroxybutyrate | 46.50 (41.15) | 30.88 (26.35) | <0.001 | Up | 1.51 | 0.005 |
| Acetic acid | 42.91 (34.69) | 50.85 (46.36) | 0.296 | Down | −1.19 | 0.570 |
| Betaine | 65.92 (57.43) | 65.78 (33.03) | 0.988 | Up | 1 | 0.989 |
| L-Carnitine | 56.83 (44.17) | 74.70 (152.08) | 0.386 | Down | −1.31 | 0.654 |
| Creatine | 43.78 (37.65) | 52.04 (33.53) | 0.214 | Down | −1.19 | 0.456 |
| Citric acid | 173.24 (132.82) | 200.36 (389.48) | 0.612 | Down | -1.16 | 0.821 |
| Choline | 12.90 (10.89) | 11.57 (7.05) | 0.443 | Up | 1.11 | 0.698 |
| D-Glucose | 6470.54 (3474.65) | 5922.48 (3311.47) | 0.386 | Up | 1.09 | 0.654 |
| Glycine | 370.29 (266.25) | 419.42 (322.74) | 0.375 | Down | −1.13 | 0.654 |
| Glycerol | 496.93 (461.91) | 455.23 (440.22) | 0.620 | Up | 1.09 | 0.823 |
| Formic acid | 31.60 (19.42) | 31.96 (15.91) | 0.911 | Down | −1.01 | 0.959 |
| L-Glutamic acid | 65.24 (42.56) | 66.92 (47.98) | 0.843 | Down | −1.03 | 0.927 |
| L-Tyrosine | 82.93 (42.46) | 88.25 (50.12) | 0.540 | Down | −1.06 | 0.769 |
| L-Phenylalanine | 88.93 (49.04) | 86.54 (42.24) | 0.779 | Up | 1.03 | 0.917 |
| L-Alanine | 484.17 (270.31) | 495.44 (250.57) | 0.816 | Down | −1.02 | 0.927 |
| L-Proline | 180.94 (81.46) | 198.18 (102.44) | 0.320 | Down | −1.1 | 0.578 |
| L-Threonine | 129.20 (100.24) | 134.59 (99.67) | 0.772 | Down | −1.04 | 0.917 |
| L-Isoleucine | 48.08 (23.12) | 50.16 (33.19) | 0.695 | Down | −1.04 | 0.855 |
| L-Histidine | 60.54 (47.15) | 62.11 (29.59) | 0.832 | Down | −1.03 | 0.927 |
| Lysine | 176.35 (109.23) | 172.42 (112.84) | 0.850 | Up | 1.02 | 0.927 |
| L-Lactic acid | 1895.02 (1159.65) | 1793.69 (914.38) | 0.601 | Up | 1.06 | 0.818 |
| Pyruvic acid | 90.23 (47.43) | 83.53 (41.57) | 0.419 | Up | 1.08 | 0.672 |
| 3-Hydroxybutyric acid | 130.52 (158.80) | 47.23 (56.47) | <0.001 | Up | 2.76 | 0.000 |
| L-Arginine | 68.61 (38.72) | 75.72 (36.96) | 0.314 | Down | −1.1 | 0.578 |
| Creatinine | 89.51 (72.30) | 84.26 (54.20) | 0.661 | Up | 1.06 | 0.843 |
| L-Glutamine | 564.41 (227.03) | 556.75 (205.71) | 0.849 | Up | 1.01 | 0.927 |
| L-Leucine | 95.59 (45.75) | 99.02 (60.58) | 0.731 | Down | −1.04 | 0.882 |
| L-Methionine | 20.11 (10.68) | 27.07 (13.65) | 0.002 | Down | −1.35 | 0.015 |
| L-Valine | 179.96 (90.10) | 187.28 (113.03) | 0.702 | Down | −1.04 | 0.855 |
| Acetone | 28.16 (24.64) | 15.71 (10.41) | <0.001 | Up | 1.79 | 0.005 |
| Methanol | 365.14 (285.28) | 338.45 (253.62) | 0.595 | Up | 1.08 | 0.817 |

In Table 5, p-value is calculated with t-test; p-value with bold and underline is calculated by the Wilcoxon Mann Whitney test.

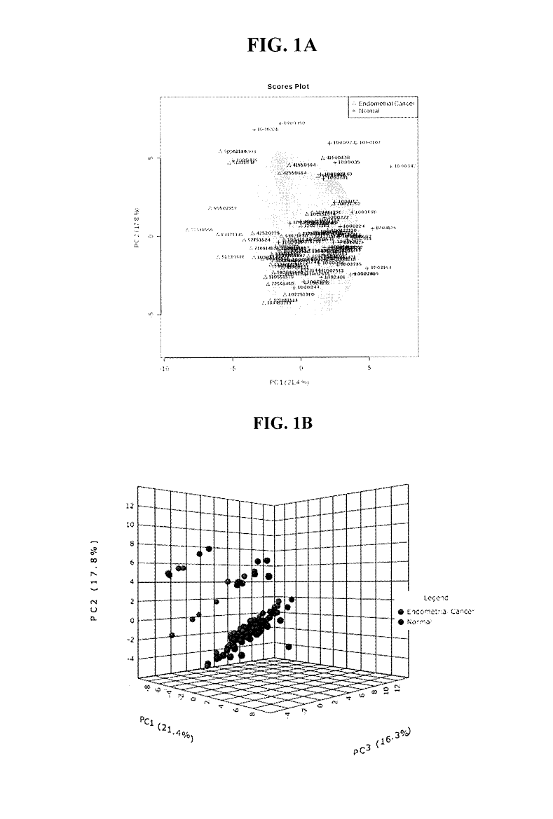

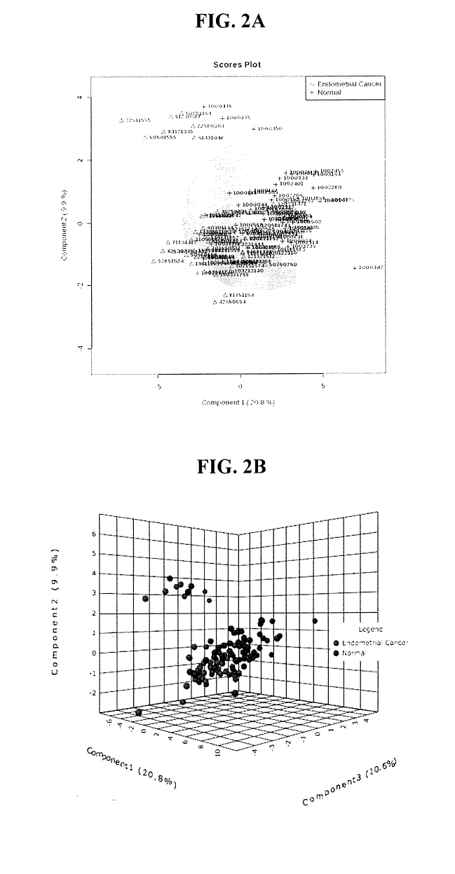

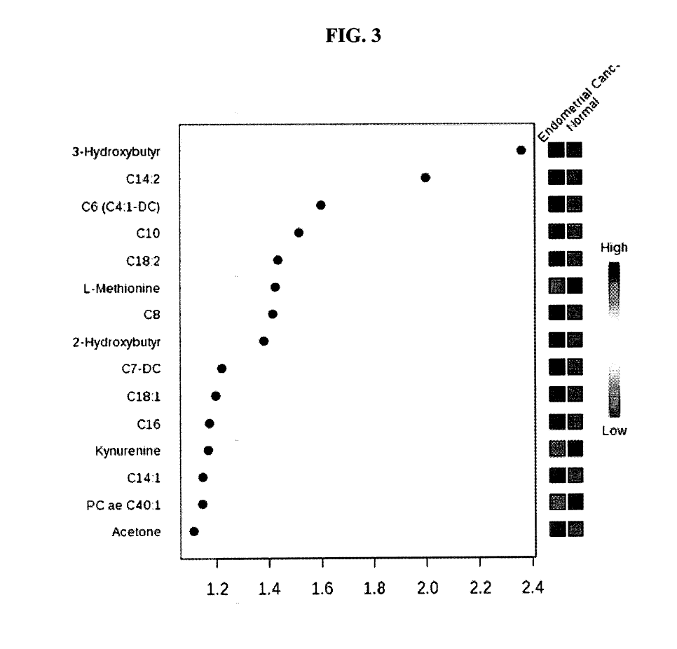

FIGS. 1A and 1B show 2-D and 3-D PCA graphs for EC overall compared to controls. Significant clustering and therefore discrimination of EC and control groups was achieved by using a combination of two and three principal components (metabolites) respectively. Further discrimination was demonstrated on PLS-DA plot (FIGS. 2A and 2B). The VIP curve ranking metabolites for their power to discriminate EC cases overall from controls, is shown in FIG. 3. The higher the VIP score, shown on the x-axis, the better the particular metabolite at distinguishing disease from the unaffected state. Permutation testing using 2000 repeat analyses were performed and yielded a p-value <0.001 indicating a less than 1 in 1,000 chance that the observed discrimination achieved by the metabolites was due to chance.

Table 6 shows the logistic regression models for the detection of EC overall using the combined NMR and DI-MS platforms. Four models were developed in the discovery group based on logistic regression analysis. These included a demographic model (a: BMI), two separate metabolite only models (b and c), and a combined metabolite and demographic (BMI) model (d) or test group using metabolites only and the predictive equation resulting from that model (model). The constituent metabolites used in the models are shown (Table 6). Those six metabolites are: C14.2, PC ae C38:1, 3-Hydroxybutyric acid, C18:2, PC ae C40:1, and C6 (C4:1-DC).

TABLE 6
Models for the Prediction of EC Overall: Combined NMR and DI-MS Metabolomic Platforms

| | | | Std. | z | | | EC/ |
|---|---|---|---|---|---|---|---|
| Model | Coefficients | Estimate | Error | value | Pr(>\|z\|) | Odds Ratio | Normal |
| a) Demographics | (Intercept) | −3.917 | 1.191 | −3.29 | 0.001 | — | — |
| only | BMI | 0.121 | 0.037 | 3.221 | 0.001 | 1.13 (1.05-1.22) | — |
| b) Metabolites | (Intercept) | −1.043 | 3.392 | −0.307 | 0.759 | — | — |
| only | C14:2 | 0.742 | 0.432 | 1.717 | 0.086 | 2.1 (0.95-5.29) | Up |
| | PC ae C38:1 | −0.761 | 0.49 | −1.552 | 0.121 | 0.47 (0.17-1.18) | Down |
| | 3-Hydroxy- | 0.69 | 0.319 | 2.164 | 0.031 | 1.99 (1.1-3.92) | Up |
| | butyric acid | | | | | | |
| c) Metabolites | (Intercept) | 15.218 | 4.041 | 3.766 | 0 | — | — |
| only | C18:2 | 2.212 | 0.958 | 2.308 | 0.021 | 9.13 (1.6-73.2) | Up |
| | PC ae C40:1 | −4.231 | 1.196 | −3.537 | 0 | 0.01 (0.0-0.11) | Down |
| | C6 (C4:1-DC) | 1.257 | 0.593 | 2.12 | 0.034 | 3.52 (1.15-12.3) | Up |
| d) Metabolites | (Intercept) | 5.875 | 2.856 | 2.057 | 0.04 | — | — |
| plus | BMI | 0.081 | 0.047 | 1.702 | 0.089 | 1.08 (1.0-1.2) | — |
| Demographics | C14:2 | 1.607 | 0.485 | 3.316 | 0.001 | 4.99 (2.15-14.82) | Up |
| | PC ae C40:1 | −3.076 | 1.125 | −2.734 | 0.006 | 0.05 (0.0-0.34) | Down |

The contribution of the constituent metabolite to prediction can be judged by the odds ratio. The performance of the respective models including area under the ROC curve, sensitivity and specificity values in the discovery group are shown. The ultimate test of the predictive accuracy of an algorithm however is how it performs in an independent test and which were not used in the original development of the model. The inventors thus evaluate these four algorithms in an independent validation group. Model performance in both the discovery and validation groups is shown in Table 7. The metabolite markers had the highest diagnostic accuracy and addition of demographic data did not improve performance of the models.

TABLE 7
Performance of Models for the Prediction of EC Overall ( Combined
NMR and DI-MS Metabolomic Platforms).

| | Discovery Group | | | 10-fold Cross-Validation | | |
|---|---|---|---|---|---|---|
| | AUC | Sensi- | Spe- | AUC | Sensi- | Spe- |
| Model* | (95% CI) | tivity | cificity | (95% CI) | tivity | cificity |
| a) Demographics | 0.757 | 0.562 | 0.806 | 0.748 | 0.545 | 0.778 |
| only | (0.719- | | | (0.630- | | |
| | 0.795) | | | 0.867) | | |
| b) Metabolites | 0.829 | 0.609 | 0.79 | 0.788 | 0.636 | 0.75 |
| | (0.798- | | | (0.682- | | |
| | 0.860) | | | 0.894) | | |
| c )Metabolites | 0.901 | 0.862 | 0.812 | 0.872 | 0.818 | 0.806 |
| | (0.877- | | | (0.783- | | |
| | 0.925) | | | 0.961) | | |
| d) Metabolites | 0.904 | 0.788 | 0.815 | 0.876 | 0.758 | 0.778 |
| and | (0.881- | | | (0.793- | | |
| demographics | 0.927) | | | 0.959) | | |

| | Validation Group | | |
|---|---|---|---|
| Model | AUC (95% CI) | Sensitivity | Specificity |
| a) Demographics | 0.663 | 0.478 | 0.792 |
| only | (0.503-0.823) | | |
| b) Metabolites | 0.826 | 0.826 | 0.708 |
| | (0.706-0.946) | | |
| c) Metabolites | 0.812 | 0.826 | 0.667 |
| | (0.687-0.936) | | |
| d) Metabolites | 0.799 | 0.783 | 0.625 |
| and | (0.672-0.926) | | |
| demographics | | | |

*See Table 6 for the actual markers used in each model

Early diagnosis and treatment of cancer is critical to significantly reducing cancer mortality, morbidities and healthcare costs. The inventors therefore evaluated the ability of metabolites to distinguish early stage EC (Stage I and II) that is confined to the uterus from controls. There were 46 early stage cancers and 60 controls. Three predictive models: BMI, metabolites only, and metabolites combined with BMI were evaluated (Table 8).

TABLE 8
Models for the Prediction of Early Stage EC* (Combined NMR and DI-MS
Metabolomic Platforms).

| | | | | | | | Early |
|---|---|---|---|---|---|---|---|
| | | | Std. | z | | | EC/ |
| Model | Coefficients | Estimate | Error | value | Pr(>\|z\|) | Odds Ratio | Normal |
| a) Demographics | (Intercept) | −4.354 | 1.29 | −3.375 | <0.001 | — | — |
| only | BMI | 0.129 | 0.04 | 3.209 | 0.001 | 1.14 (1.06-1.24) | — |
| b) Metabolites | (Intercept) | −1.446 | 3.688 | −0.392 | 0.695 | — | — |
| only | C14.2 | 0.814 | 0.478 | 1.703 | 0.089 | 2.26 (0.95-6.4) | Up |
| | PC ae C38:1 | −0.986 | 0.527 | −1.87 | 0.062 | 0.37 (0.12-1.0) | Down |
| | 3-Hydroxy- | 0.742 | 0.34 | 2.184 | 0.029 | 2.10 (1.12-4.34) | Up |
| | butyric acid | | | | | | |
| c) Metabolites | (Intercept) | 6.867 | 3.26 | 2.106 | 0.035 | — | — |
| plus | BMI | 0.1 | 0.053 | 1.874 | 0.061 | 1.1 (1.01-1.24) | — |
| demographics | C14:2 | 1.975 | 0.603 | 3.274 | 0.001 | 7.2 (2.61-29.3) | Up |
| | PC ae C40:1 | −3.184 | 1.272 | −2.502 | 0.012 | 0.04 (0.0-0.39) | Down |

*Early EC versus no Cancer

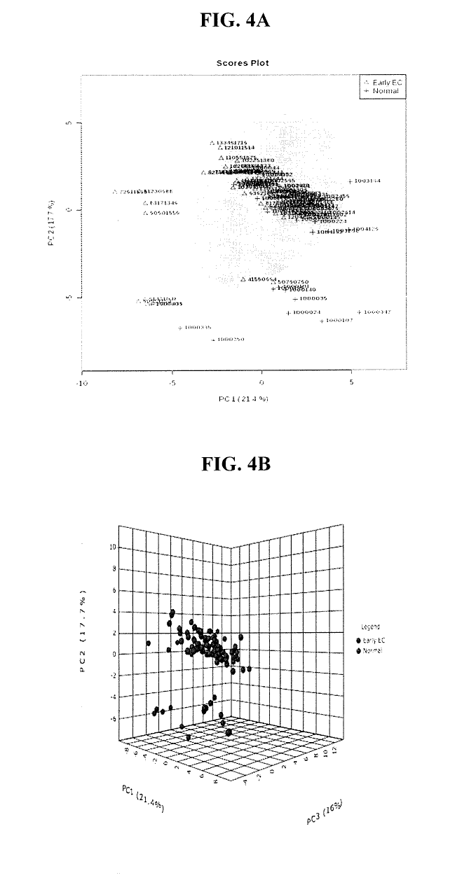

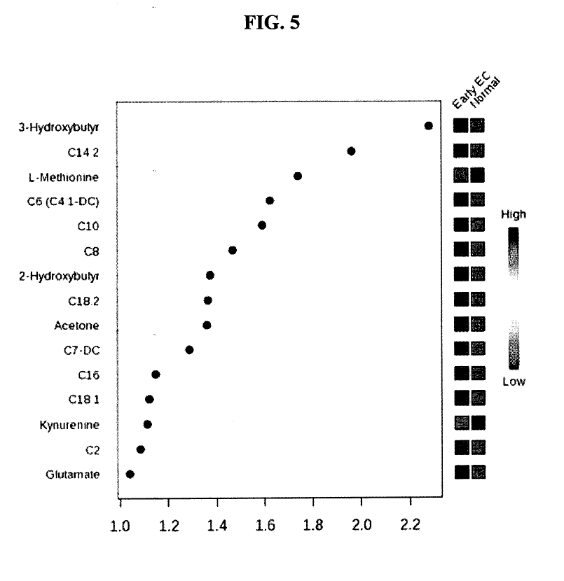

FIGS. 4A and 4B show the 2-D and 3-D PCA plots for early stage EC compared to controls (normal). Significant clustering and therefore discrimination of early EC and control groups was achieved by using two and three principal components (metabolite combinations), respectively. FIG. 5 shows the VIP curve ranking of metabolites for their power to discriminate early EC from controls. The direction of change of these metabolites is also indicated on the VIP plot. Permutation testing using 2000 repeat analyses were performed and yielded a p-value <0.001 indicating a less than 1 in 1,000 chance that the observed discrimination achieved by the metabolites was due to chance. The performance of the three models for early EC detection is shown Table 9. The metabolites significantly detect early EC independent of BMI.

TABLE 9
Performance of Models for the Prediction of Early Stage EC (Combined
NMR and DI-MS Metabolomic Platforms).

| | Discovery Data | | | 10 fold Cross Validation | | |
|---|---|---|---|---|---|---|
| | AUC | Sensi- | Spe- | AUC | Sensi- | Spe- |
| Model | (95% CI) | tivity | cificity | (95% CI) | tivity | cificity |
| a) Demographics | 0.769 | 0.508 | 0.849 | 0.762 | 0.500 | 0.806 |
| | (0.730- | | | (0.640- | | |
| | 0.808) | | | 0.884) | | |
| b) Metabolites | 0.848 | 0.627 | 0.830 | 0.814 | 0.607 | 0.806 |
| | (0.817- | | | (0.710- | | |
| | 0.878) | | | 0.917) | | |
| c) Metabolites | 0.924 | 0.790 | 0.833 | 0.906 | 0.821 | 0.833 |
| and | (0.903- | | | (0.832- | | |
| demographics | 0.945) | | | 0.980) | | |

| | Validation Data | | |
|---|---|---|---|
| Model | AUC (95% CI) | Sensitivity | Specificity |
| a) Demographics | 0.616 | 0.333 | 0.833 |
| | (0.438-0.793) | | |
| b) Metabolites | 0.819 | 0.722 | 0.792 |
| | (0.689-0.950) | | |
| c) Metabolites | 0.799 | 0.722 | 0.750 |
| and | (0.665-0.932) | | |
| demographics | | | |

*See Table 8 for the actual markers used in each model.

PCA analysis was performed to determine the ability of the metabolites to distinguish Stage I and II EC, from Stage III and IV disease and unaffected controls (not shown). Permutation testing revealed that metabolites significantly distinguished these groups (p<0.001). This was mainly predicated on the ability to distinguish EC cases from controls. The inventors subsequently performed PCA and PLS-DA analysis (not shown) to see whether the metabolites could distinguish early from late stage EC. The plots did show clustering or discrimination between these two groups, however permutation testing found that the discrimination was not statistically significant (p-value=0.308). The inventors believe the lack of significance was plausibly due to the small number of late stage EC cases and consequent lack of study power.

Finally, BMI is known to be a significant risk factor for EC. The inventors therefore evaluated the correlation between BMI and important predictive metabolites (see, Table 10).

TABLE 10
Pairwise Correlation Analysis for the selected metabolites with BMI.

| Correlation | | | PC ae | 3-Hydroxy- | | PC ae | C6(C4:1- |
|---|---|---|---|---|---|---|---|
| p-value | BMI | C14:2 | C38:1 | butyric acid | C18:2 | C40:1 | DC) |
| BMI | 1.000 | | | | | | |
| C14:2 | 0.093 | 1.000 | | | | | |
| | 1.000 | | | | | | |
| PC ae C38:1 | −0.229 | −0.107 | 1.000 | | | | |
| | 0.281 | 1.000 | | | | | |
| 3-Hydroxy- | 0.098 | 0.442* | −0.053 | 1.000 | | | |
| butyric acid | 1.000 | 0.000 | 1.000 | | | | |
| C18:2 | 0.127 | 0.768* | −0.028 | 0.384* | 1.000 | | |
| | 1.000 | 0.000 | 1.000 | 0.000 | | | |
| PC ae C40:1 | −0.255 | −0.137 | 0.4736* | −0.195 | −0.011 | 1.000 | |
| | 0.121 | 1.000 | 0.000 | 0.763 | 1.000 | | |
| C6(C4:1-DC) | 0.156 | 0.665* | 0.013 | 0.496* | 0.553* | −0.113 | 1.000 |
| | 1.000 | 0.000 | 1.000 | 0.000 | 0.000 | 1.000 | |

*p-value < 0.05; this was performed with pairwise correlation analysis with Bonferroni correction; there was no significant correlation between BMI and metabolites

Overall therefore metabolite markers appear to be strong predictors of EC overall and also of early EC. Based on regression analysis, traditional demographic risk factors such as age, race, diabetes status and BMI did not meaningfully add to the diagnostic performance of the metabolite markers.

What is claimed is:

1. A method of detecting a level of two or more metabolites in a biological sample, said method consisting of:
obtaining a biological sample from a human patient, wherein said biological sample includes two or more of C14.2, PC ae C38:1, 3-Hydroxybutyric acid, C18:2, PC ae C40:1, and C6 (C4:1-DC); and
detecting the level of the two or more metabolites in the biological sample.

2. The method of claim 1, wherein the sample is blood serum.

3. The method of claim 1, wherein the two or more metabolites are C14.2, PC ae C38:1, and 3-Hydroxybutyric acid.

4. The method of claim 1, wherein the two or more metabolites are C18:2, PC ae C40:1, and C6 (C4:1-DC).

5. The method of claim 1, wherein the level of the two or more metabolites are detected by performing Magnetic Resonance spectroscopy (MRS) using a magnetic resonance imaging (MRI) machine, nuclear magnetic resonance (NMR), or mass spectrometry (MS) on the biological sample.

6. The method of claim 5 wherein the two or more metabolites are detected by magnetic resonance spectroscopy (MRS).

7. The method of claim 6, wherein the MRS is proton magnetic resonance spectroscopy (1H-MRS)

8. A method of diagnosing endometrial cancer in a human patient, wherein said human patient has a uterus with an endometrium, said method comprising:
obtaining a biological sample from the human patient, wherein said biological sample includes one or more metabolites C14.2, PC ae C38:1, 3-Hydroxybutyric acid, C18:2, PC ae C40:1, and C6 (C4:1-DC);
detecting a level of the one or more metabolites in the biological sample;
performing an ultrasound of the uterus to determine the thickness of the endometrium of the patient; and
diagnosing the patient with endometrial cancer when (a) the one or more metabolites in the biological sample is at a different level than a statistically validated threshold for the one or more metabolites and (b) the ultrasound indicates endometrial cancer in the patient.

9. The method of claim 8, wherein the level of the one or more metabolites are detected by performing Magnetic Resonance spectroscopy (MRS) using a magnetic resonance imaging (MRI) device, nuclear magnetic resonance (NMR), or mass spectrometry (MS) on the biological sample.

10. The method of claim 9 wherein the one or more metabolites are detected by magnetic resonance spectroscopy (MRS).

11. The method of claim 10, wherein the MRS is proton magnetic resonance spectroscopy (1H-MRS)

12. The method of claim 8, wherein the sample is blood serum.

13. The method of claim 8, wherein the one or more metabolites are C14.2, PC ae C38:1, and 3-Hydroxybutyric acid.

14. The method of claim 8, wherein the one or more metabolites are C18:2, PC ae C40:1, and C6 (C4:1-DC).

15. A method of diagnosing and treating endometrial cancer in a subject, said method comprising:
obtaining a biological sample from the human subject, wherein said biological sample includes one or more metabolites C14.2, PC ae C38:1, 3-Hydroxybutyric acid, C18:2, PC ae C40:1, and C6 (C4:1-DC);
detecting a level of the one or more metabolites in the biological sample;
diagnosing the subject with endometrial cancer when the one or more metabolites in the biological sample is at a different level than a statistically validated threshold for the one or more metabolites; and
administering a therapeutically effective amount of a treatment for endometrial cancer to the diagnosed subject.

16. The method of claim 15, wherein the treatment is surgery, radiation, or chemotherapy.

17. The method of claim 15, wherein the level of the one or more metabolites are detected by performing Magnetic Resonance spectroscopy (MRS) using a magnetic resonance imaging (MRI) device, nuclear magnetic resonance (NMR), or mass spectrometry (MS) on the biological sample.

18. The method of claim 17 wherein the one or more metabolites are detected by magnetic resonance spectroscopy (MRS).

19. The method of claim 18, wherein the MRS is proton magnetic resonance spectroscopy (1H-MRS)

20. The method of claim 15, wherein the sample is blood serum.

21. The method of claim 15, wherein the one or more metabolites are C14.2, PC ae C38:1, and 3-Hydroxybutyric acid.

22. The method of claim 15, wherein the one or more metabolites are C18:2, PC ae C40:1, and C6 (C4:1-DC).

23. A method of providing medical services for a human patient suspected of having or having endometrial cancer, said method comprising:
requesting a biological sample from and diagnostic information about the patient, wherein the diagnostic information is a level of one or more metabolites C14.2, PC ae C38:1, 3-Hydroxybutyric acid, C18:2, PC ae C40:1, and C6 (C4:1-DC) in the biological sample; and
administering a therapeutically effective amount of a treatment for endometrial cancer when the diagnostic information indicates that the level of the one or more metabolites in the biological sample is at a different level than a statistically validated threshold for the one or more metabolites.

24. The method of claim 23, wherein the treatment is surgery, radiation, or chemotherapy.

25. The method of claim 23, wherein the diagnostic information is determined by Magnetic Resonance spectroscopy (MRS) using a magnetic resonance imaging (MRI) device, nuclear magnetic resonance (NMR), or mass spectrometry (MS) on the biological sample.

26. The method of claim 23, wherein the sample is blood serum.

27. The method of claim 23, wherein the one or more metabolites are C14.2, PC ae C38:1, and 3-Hydroxybutyric acid.

28. The method of claim 23, wherein the one or more metabolites C18:2, PC ae C40:1, and C6 (C4:1-DC).

29. A method of monitoring treatment for endometrial cancer in a human patient, comprising:
requesting a first biological sample from and first diagnostic information about the patient, wherein the first diagnostic information is a level of one or more metabolites C14.2, PC ae C38:1, 3-Hydroxybutyric acid, C18:2, PC ae C40:1, and C6 (C4:1-DC) in the first biological sample;
administering a therapeutically effective amount of a treatment for endometrial cancer to the patient;
after administering the therapeutically effective amount of the treatment for endometrial cancer to the patient, requesting a second biological sample from and second diagnostic information about the patient, wherein the second diagnostic information is a level of one or more metabolites C14.2, PC ae C38:1, 3-Hydroxybutyric acid, C18:2, PC ae C40:1, and C6 (C4:1-DC) in the second biological sample; and
comparing the first diagnostic information and the second diagnostic information to determine whether the level of the one or more metabolites in the first biological sample is at a different level than the level of the one or more metabolites in the second biological sample.

30. The method of claim 29, wherein the sample is blood serum.
